(19) United States
(12) Patent Application Publication
(10) Pub. No.: US 2022/0332770 A1
(43) Pub. Date: Oct. 20, 2022

(54) High-Density Flagellin-Displaying Virus-Like Particle As Vaccine Carrier

(71) Applicant: University of Rhode Island Board of Trustees, Kingston, RI (US)

(72) Inventors: Xinyuan CHEN, East Greenwich, RI (US); Yiwen ZHAO, South Kingstown, RI (US)

(21) Appl. No.: 17/719,015

(22) Filed: Apr. 12, 2022

Related U.S. Application Data
(60) Provisional application No. 63/173,994, filed on Apr. 12, 2021.
(60) Provisional application No. 63/277,011, filed on Nov. 8, 2021.

(51) Int. Cl. C07K 14/255; C07K 14/005; A61K 39/39; A61K 39/145; A61P 31/16; C12N 7/00; A61K 39/00; A61P 37/04; A61P 25/34

(52) U.S. Cl. CPC C07K 14/255; C07K 14/005; A61K 39/39; A61K 39/145; A61P 31/16; C12N 7/00; A61K 39/0011; A61P 37/04; A61P 25/34; A61K 39/0013; C07K 2319/33; C12N 2760/16071; C12N 2760/16034; A61K 2039/5258; A61K 2039/6068; C12N 2760/16022

(57) ABSTRACT

The invention provides a novel fusion protein between flagellin (or portions thereof) and a polypeptide that can form a virus-like particle (VLP) (e.g., hepatitis b core (HBc) protein or portions thereof), where the fusion protein continues to form a VLP in an aqueous environment. The VLPs based on such fusion proteins (e.g., FH VLPs) provide a versatile, highly immunogenic, and safe vaccine carrier capable of displaying or associating a variety of vaccine antigens on VLP surface to elicit potent humoral and cellular immune responses.

### CROSS-REFERENCE TO RELATED APPLICATIONS

This application claims priority to and the benefit of co-pending U.S. provisional patent applications Ser. Nos. 63/173,994 and 63/277,011, filed Apr. 12, 2021 and Nov. 8, 2021, respectively, which applications are incorporated herein by reference in entirety.

### REFERENCE TO SEQUENCE LISTING

Sequence listings and related materials in the ASCII text file named “SEQ-URI2102 ST25.txt” and created on Apr. 11, 2022 with a size of about 78 kilobytes, is hereby incorporated by reference.

### STATEMENT REGARDING FEDERALLY SPONSORED RESEARCH OR DEVELOPMENT

This invention was made with government support under grant number AI156510 awarded by the National Institutes of Health. The Government has certain rights in the invention.

### FIELD OF THE INVENTION

The invention relates to virus-like particle (VLP)-based vaccines, vaccine delivery and vaccine adjuvants in general and particularly to a new vaccine platform that displays flagellin at high densities on virus-like particle (VLP) surface to greatly reduce any systemic adverse effects from flagellin-based vaccines while providing improved immunogenicity and efficacy.

### BACKGROUND OF THE INVENTION

Flagellin is the principal component of flagellar filament found in flagellated bacteria such as Salmonella, Escherichia coli, Vibrio vulnificus, Campylobacter coli, and P. aeruginosa. The flagellin protein typically has four structural domains (D0, D1, D2, and D3). D0 and D1 domains from the inner filament core and are composed of highly conserved N- and C-termini of flagellin in the form of α-helical coiled coils (FIG. 1). D2 and D3 domains form the outer surface of filament and are composed of highly variable central region of flagellin (see, K. Yonekura, et al., Nature 2003, 424(6949): 643-50).

Extracellular flagellin can be recognized by toll-like receptor (TLR) 5 and intracellular flagellin can be recognized by NOD-like receptor (NLR) family, apoptosis inhibitory protein 5 (NAIP5) to initiate NLR family CARD domain containing 4 (NLRC4) inflammasome activation (see, F. Hayashi, et al., Nature 2001, 410(6832): 1099-103; Y. Zhao, et al., Nature 2011, 477(7366): 596-600). D1 domain contributes to TLR5 binding and filament formation and D0 domain contributes to NAIP5 recognition and NLRC4 inflammasome activation (see, e.g., W. S. Song, et al., Sci. Rep. 2017, 7: 40878). TLR5 binding leads to NFKB activation and release of pro-inflammatory cytokines, such as interleukin (IL)-6, while NLRC4 inflammasome activation leads to Caspase-1 activation (M. Vijay-Kumar, et al., Eur. J. Immunol. 2010, 40(12): 3528-34).

Flagellin has been explored as vaccine adjuvants and carriers as it appears to activate both the innate and adaptive arms of immunity and therefore holds great promise as highly immunogenic vaccine carriers when compared to other vaccine approaches (see, I.A. Hajam, et al., Exp. Mol. Med. 2017, 49(9): e373). FljB and FliC are two closely related but alternately expressed forms of flagellin in gram-negative Salmonella enterica serovar Typhimurium (S. Typhimurium) (see, H. R. Bonifield, et al., J. Bacteriol. 2003, 185(12): 3567-74), and indeed both have been explored for vaccine development.

Despite being highly immunogenic, two flagellin-based influenza vaccines (VAX125 and VAX102) induced significant systemic adverse reactions especially at high doses in clinical studies (J. J. Treanor, et al., Vaccine 2010, 28(52): 8268-74; C.B. Turley, et al., Vaccine 2011, 29(32): 5145-52). Specifically, both vaccines dose-dependently increased serum c-reactive protein (CRP) levels and, in some patients, systemic adverse reactions were accompanied with elevated serum IL-6 levels. Another flagellin-based influenza vaccine (VAX128) also dose-dependently increased serum CRP levels in the elderly and 4.8% subjects experienced at least 4-fold increase of serum IL-6 (D. N. Taylor, et al., Vaccine 2012, 30(39): 5761-9). Increase of serum CRP and IL-6 levels were also observed in a recent clinical trial of a different flagellin-based influenza vaccine (VAX2012Q) (L. Tussey, et al., Open Forum Infect. Dis. 2016, 3(1): ofw015). Considering TLR5 activation leads to IL-6 secretion that in turn activates CRP release, over activation of TLR5 on various types of lymphoid and non-lymphoid cells might explain, at least partly, systemic adverse reactions such as cytokine storm and fever that have been associated, without exception, with existing flagellin-based vaccine candidates (e.g., S. B. Mizel, et al., J. Immunol. 2010, 185(10): 5677-82).

Therefore, despite flagellin's great promise as a potential vaccine carrier, its high risk for inducing strong side effects has posed safety concerns that prevented it from surviving a clinical trial and making to the pharmaceutical market so far. Accordingly, there remains a strong need to develop safer flagellin-based yet highly immunogenic vaccine carriers.

Meanwhile, in pursuit of safer vaccination, researchers have turned to subunit vaccines more and more due to their significantly improved safety profiles as compared to whole pathogen-based vaccines. Yet, subunit vaccines have relatively low immunogenicity because of their weak uptake by antigen-presenting cells (APCs) and weak ability to stimulate APC activation. Efficient antigen uptake and stimulation of APC maturation are crucial to induce potent antigen-specific immune responses (R.M. Steinman, et al., Current Topics in Microbiology and Immunology 2006, 311: 17-58). Besides infectious disease vaccines, cancer vaccines based on tumor-associated antigens (TAAs) and neoantigens that are mainly presented on major histocompatibility complex (MHC) class II molecules (see, E. Blass, et al., Nat Rev Clin Oncol 2021, 18(4): 215-229). Failing to induce APC maturation and potent cytotoxic T lymphocyte (CTL) responses have presented significant challenges for the successful adoption of subunit vaccine technologies.

Vaccine delivery platforms facilitate antigen uptake by APCs and can be used to enhance subunit vaccine efficacy. Highly immunogenic proteins and virus-like particles (VLPs) are both popular vaccine delivery platforms (J. Wallis, et al., Clin Exp Immunol 2019, 196(2): 189-204). VLPs are self-assembled nanostructures, mimicking the size and shape of infectious viruses. VLPs can be presented on both MHC I and II molecules and elicit both humoral and cellular immune responses (see K. M. Frietze, et al., Curr Opin Virol 2016, 18: 44-9). Some VLPs can be used as vaccines alone, such as hepatitis b surface antigen (HBsAg), while others can be used to display vaccine antigens from other pathogens to serve as vaccine delivery platforms, such as Hepatitis B core (HBc) VLPs.

HBc self-assembles into VLPs and the c/el loop of HBc provides a site for high-density display of antigenic epitopes on VLP surface (e.g., U. Arora, et al., J. Nanobiotechnol. 2012, 10: 30). However, the c/e1 loop of HBc, localized at the tip of its spikes, allows the insertion of only short antigenic epitopes without imposing significant steric hindrance to negatively impact HBc VLP assembly (K. Roose, et al., Expert Review of Vaccines 2013, 12(2): 183-98). As of now, only two full-length proteins, green fluorescence protein (GFP, 238 aa) and outer surface protein C of the Lyme disease agent Borrelia burgdorferi (OspC, 189 aa) with closely juxtaposed N and C-termini, have been successfully displayed on HBc VLP surface via c/el loop insertion (P. A. Kratz, et al., Proc. Natl. Acad. Sci. U.S.A. 1999, 96(5): 1915-20; C. Skamel, et al., J. Biol Chem. 2006, 281(25): 17474-81). No success has been reported with recombinantly displaying a protein or fragments thereof with a size larger than 250 amino acids on HBc VLP surface. This shortcoming has greatly hampered the use of VLPs as a vaccine delivery platform.

### SUMMARY OF THE INVENTION

The present invention, through unexpectedly successful display of flagellin or one or more portions thereof on a VLP surface, in a high-density fashion, provides a versatile, highly immunogenic, and surprisingly safe vaccine platform. The full-length flagellin protein is well over 450 amino acid residues and its incorporation would normally be expected to have hindered the proper folding and assembly of existing VLP platforms, and the expression of such a highly immunogenic molecule or even portions thereof would have also been expected to induce severe systemic adverse reactions in the body. However, due to unique structural geometries and clever engineering, the flagellin-displaying VLP platform of the invention turned out to be well tolerated and even more immunogenic than many existing vaccine carriers.

The present invention can be used to aid in vaccine development against emerging infectious diseases, such as COVID-19, or develop better, even universal, vaccines against known pathogens that mutate frequently such as the ever-evolving strains of influenza and other viruses. This is of particular public health importance since the current influenza vaccines vary from year to year in efficacy and induce only weak protection in the very young and the elderly. The platform based on the present invention can be used in many different ways: to make or deliver vaccines or immunogens, e.g., through recombinantly incorporating or chemically conjugating to an antigenic epitope or a full-length antigen (or a portion thereof); or to function as an adjuvant to boost the body's immune reaction when co-administered with an immunogenic agent, e.g., an existing vaccine.

In one aspect of the invention, the high-density display of flagellin proteins (or a portion thereof) on the VLP surface likely has successfully embedded the flagellin protein's D0 and D1 domains, or substantial portions thereof, in the interior of VLP and thus reduced flagellin's TLR5 activation ability and improved the systemic safety. At the same time, transition of flagellin from soluble to particulate form on VLPs may explain the increased immunogenicity that was recorded. In a further aspect of the invention, the D3 domain (and possibly the D2 domain as well) of flagellin which extends the furthest out on the VLP surface, allows recombinant insertion of anything ranging from small epitopes to large antigens without interfering or jeopardizing the VLP assembly. In yet another further aspect, part or all of the D3 domain and, optionally, part or all of the D2 domain of flagellin are replaced by a desired immunogen with little restrictions on the size and tertiary structure of such immunogen.

In one aspect, the invention relates to a fusion protein comprising parts as follows: (a) the amino acid sequence for the entire or substantial portions of two or more of the four domains of a flagellin protein, preferably the D0 and D1 domains or substantial portions thereof, and (b) the amino acid sequence for a polypeptide that can form a virus-like particle (VLP) (a “VLP-forming polypeptide”) in aggregate, wherein (a) and (b) are recombinantly linked to each other. The novel fusion protein preferably self-assembles with other such fusion proteins and forms a novel VLP in an aqueous environment (e.g., in a saline solution such as phosphate-buffered saline (PBS) or serum). A preferred embodiment of the VLP-forming polypeptide for constructing the fusion protein of the invention is a Hepatitis B core (HBc) protein or a substantial portion thereof. The fusion protein according to the present invention can further include: (c) an immunogenic amino acid sequence, e.g., an epitope or a full-length antigen, that serves as an immunogen such as an influenza epitope, a tumor-associated antigen (TAA) or a neoantigen. Alternately, the immunogen can be a molecule or compound chemically conjugated to the fusion protein having parts (a) and (b). Such immunogen can be a polysaccharide-based, hapten-based agent, e.g., a nicotine hapten molecule like a carboxymethlureido-nicotine molecule. The fusion protein of the invention, in the form of a self-assembled VLP, can be used as a vaccine, a vaccine carrier or a vaccine adjuvant.

In a particularly preferred embodiment, the present invention provides a flagellin-HBc fusion protein comprising the amino acid sequence of a full-length flagellin recombinantly inserted into any position within, or replacing part or all of, the amino acid sequence of the c/e1 loop (N75-L84) of a hepatitis b core (HBc) protein. The embodiment may further include at least one immunogenic sequence, such as a protein or a portion thereof found in an influenza virus, a sever acute respiratory syndrome (SARS) coronavirus, and so on. Other examples of such immunogenic sequences include an antigen expressed by a cancer or other diseased cell, e.g., tumor-associated antigens or neoantigens or a portion thereof. In an embodiment, the immunogenic sequence is recombinantly inserted into the amino acid sequence for the D2 or D3 domain of flagellin or recombinantly inserted by replacing part or entire D2, D3 or both D2 and D3 domains of flagellin. Exemplary embodiments include: (1) where D0 and D1 domains of FljB, flanked with optional short linker sequences, are inserted to replace the entire c/el loop of a truncated HBc (SEQ ID NO:28); and (2) where D0, D1, and D2 domains of FljB, flanked with optional short linker sequences, are inserted to replace the entire c/el loop of a truncated HBc (SEQ ID NO:29). Alternately, the invention provides a fusion protein with sequences for both flagellin and HBc (or substantial portions thereof) where the fusion protein is chemically conjugated to an immunogenic compound or biomolecule.

In one feature, the invention provides a fusion protein having amino acid sequences for (a) an antigen or immunogen, (b) a flagellin protein or a substantial portion thereof, (c) a virus-like particle (VLP)-forming polypeptide, where (a) (b) and (c) are recombinantly linked. In various embodiments, the fusion protein includes the amino acid sequence selected from the group of SEQ ID NOs: 23, 24, 27, 28 and 29, or a substantial portion thereof that is capable of self-assemble with the same or similar polypeptides into a virus-like particle (VLP) in an aqueous environment.

In one aspect, the present invention provides a virus-like particle (VLP) comprising an assembly of proteins, each being a fusion protein described herein.

In a further aspect, the invention provides genetic materials including polynucleotides that, when expressed, produce the fusion proteins of the invention. Such genetic materials include and are not limited to DNAs and RNAs including mRNA.

In another aspect, the invention provides a complex comprising a flagellin-containing VLP conjugated to an immunogen or antigenic agent such as a polysaccharide-based, hapten-based agent, e.g., a nicotine hapten molecule like a carboxymethlureido-nicotine molecule.

In another aspect, the invention features a virus-like particle (VLP) in an aqueous solution where the VLP displays at least 100, preferably at least 150, 180, or 240 full-length flagellin proteins or substantial portions thereof on an outer surface of the VLP.

Further aspects of the invention include a pharmaceutical composition, manufacture and use thereof where the composition comprises (a) a fusion protein, complex or VLP described herein, and (b) a pharmaceutically acceptable carrier, additive or excipient. Such compositions, when used as a vaccine, can optionally include at least an adjuvant such as cytosine phosphoguanine (CpG), a synthetic form of DNA that mimics bacterial and viral genetic material and aluminum-containing adjuvants (Alum). Other examples of adjuvants that can be used in conjunction with pharmaceutical compositions disclosed herein include squalene emulsion based MF59 and AS03 adjuvant, monophosphoryl lipid A (MPL) and so on. A method of treating a patient in need thereof by administering such a pharmaceutical composition is also part of the invention.

Preferred embodiments of the invention include vaccines against infectious pathogens such as an influenza vaccine that includes amino acid sequences for (a) an antigenic or immunogenic sequence against one or more influenza strains, (b) a flagellin protein or a substantial portion thereof, (c) a virus-like particle (VLP)-forming polypeptide or a substantial portion thereof, wherein (a) (b) and (c) are recombinantly linked. A preferred embodiment of the influenza vaccine is called a universal influenza vaccine that is effective against multiple influenza strains.

Another preferred embodiment of the invention is a cancer vaccine that includes amino acid sequences for: (a) an antigenic sequence expressed on a cancer cell, (b) a flagellin protein or a substantial portion thereof, (c) a virus-like particle (VLP)-forming polypeptide or a substantial portion thereof, where (a) (b) and (c) are recombinantly linked. Targeted cancers, in one embodiment, include: melanoma, breast cancer, and prostate cancer.

Another aspect of the invention relates to a method of making or using the VLPs of the invention: (a) using as an adjuvant with another antigenic or immunogenic agent, which can be any type of vaccine (e.g., recombinant, conjugated, VLP-based, toxoid-based, subunit-based, and so on); (b) making a vaccine by conjugating to an immunogenic agent; (c) making a vaccine by recombinantly inserting an immunogenic sequence into a fusion protein that forms VLP.

The foregoing and other objects, aspects, features, and advantages of the invention will become more apparent from the following description and from the claims.

### BRIEF DESCRIPTION OF THE DRAWINGS

The patent or application file contains at least one drawing executed in color. Copies of this patent or patent application publication with color drawings will be provided by the Office upon request and payment of the necessary fee.

The objects and features of the invention can be better understood with reference to the drawings described below, and the claims. The drawings are not necessarily to scale, emphasis instead generally being placed upon illustrating the principles of the invention. In the drawings, like numerals are used to indicate like parts throughout the various views.

FIG. 1A illustrates the crystal structure of a flagellin protein (FliC, PDB: 1UCU) in web-based 3D structure viewer (iCN3D). The N- and C-termini are labeled with letters “N” and “C,” respectively, and the four domains are also indicated. FIG. 1B shows the amino acid sequences of the FljB (SEQ ID NO:1) and FliC (SEQ ID NO:2) proteins of Salmonella enterica serovar Typhimurium (S. Typhimurium) strain LT2. FIG. 1C illustrates how the structure shown in FIG. 1A was used to measure molecular lengths. Distances between Leu493 (end of D0 domain) and Asn406 (middle of D1 and D2 domains), between Leu493 and Ala184 (middle of D2/D3 domains), and between Leu493 and Gly240 (end of D3 domain) were illustrated.

FIG. 2 illustrates protein sequence alignment between S. Typhimurium FljB (GenBank Id: AAC94993.1, SEQ ID NO:1) and FliC (GenBank Id: ABJ98783.1, SEQ ID NO:2). Online protein Blast (U.S. National Library of Medicine) was used to align FljB (top) and FliC (bottom) protein sequences. Consensus sequence (SEQ ID NO:3) is listed as a separate line in the middle.

FIGS. 3A and 3B show result of sequence alignment, using CLUSTAL O (1.2.4), between variants of flagellin proteins across the following species with their respective GenBank id listed first:
AF045151.1 Salmonella typhimurium phase 2 flagellin (fljB) (SEQ ID NO:8);
EF057754.1 Salmonella typhimurium phase I flagellin middle domain variant C108 (fliC) (SEQ ID NO:9);
U05298.1 Salmonella danysz phase-1 flagellin (fliC) (SEQ ID NO:10);
M64671.1 C.coli flagellin (flaA and flaB) (SEQ ID NO:11);
M57501.1 Pseudomonas aeruginosa flagellin (flaA) (SEQ ID NO:12);
U54775.1 Pseudomonas aeruginosa flagellin (fliC) and FlaG (flaG) (SEQ ID NO:13); and
JX847136.1 Escherichia coli strain MM_1857 FliC (fliC) (SEQ ID NO:14).

Other strains that are not presented here include and are not limited to: BA_{000037.2} Vibrio vulnificus major flagellin (FlaB), and so on.

FIG. 4 illustrates the crystal structure of HBc protein in web-based 3D structure viewer (iCN3D). Protein data bank (PDB) codes used was 1QGT. The c/el loop (N75-L84) (SEQ ID NO:15) of HBc is highlighted in magenta.

FIG. 5 schematically illustrates the VLP structure of the invention where flagellin or at least a substantial portion thereof is displayed in high-density on a VLP such as HBc VLP.

FIGS. 6A-6D illustrate the expression and characterization of recombinant FljB-HBc, FljB and HBc as described in the examples. FIG. 6A: Schematic of DNA construct for FljB-HBc fusion protein expression. Full-length FljB (1-506, SEQ ID NO:1), flanked with optional (G45)2 linkers, was inserted between A₈₀ and S81 of c/el loop of a truncated HBc (1-149, Adw subtype, SEQ ID NO:16), the resulting full sequence for this illustrative embodiment of a fusion protein of the invention is SEQ ID NO:27 (referred to as “FH” or “FH VLP” when assembled into a VLP). FIG. 6B: Recombinant FljB-HBc, FljB, and HBc were subjected to SDS-PAGE analysis. FIGS. 6C and 6D: Recombinant FljB-HBc, FljB, and HBc were subjected to western blotting analysis. Anti-FljB antiserum was used to blot PVDF membrane in FIG. 6C and anti-HBc antiserum was used to blot PVDF membrane in FIG. 6D.

FIGS. 7A-7D illustrate the characterization of FljB-HBc (FH) VLPs and HBc VLPs. FIG. 7A: Dynamic light scattering (DLS) and zeta potential measurements of FH and HBc VLPs. Particle size distributions were shown in the left. Particle size, polydispersity index (PDI), and zeta potential were listed on the right. FIG. 7B: Representative transmission electron microscope (TEM) images of FH and HBc VLPs following negative staining. Arrows point to clearly small particles. Scale: 50 nm. FIG. 7C: Representative TEM images of FH VLPs following immunogold staining with anti-FljB antiserum (left) or non-immune serum (middle) or HBc VLPs following immunogold staining with anti-FljB antiserum (right). Arrows point to VLPs. Scale: 50 nm. FIG. 7D: Particle size of FH and HBc VLPs after immunogold staining as shown in FIG. 7C was measured in Image J (n=32-46). One-way ANOVA with Tukey's multiple comparison test was used to compare differences between groups in FIG. 7D. ***: p<0.001.

FIGS. 8A-8D show data that indicate impaired TLR5 and Caspase-1 activation by FH VLPs. FIG. 8A: TLR5 activation ability of FH VLPs, FljB, and FLA-ST (highly purified FliC from Salmonella typhimurium, Invivogen). HEK293 cells co-transfected with TLR5 and SEAP reporter gene were incubated with FH VLPs, FljB, and FLA-ST in triplicate at 8, 40, 200, 1000 pM of respective proteins to keep equal flagellin contents across groups at each treatment concentration. OD62onm was read 10 hours later to indicate relative TLR5 activation ability. FIG. 8B: Caspase-1 activation ability of FH VLPs, FljB, and FLA-ST. Bone marrow-derived dendritic cells (BMDCs) were incubated with FH VLPs, FljB, and FLA-ST at 70 nM of respective protein levels in duplicate for 24 hours. Expression of pro-Caspase-1, Caspase-1 (p20), and house-keeping gene GAPDH was analyzed by western blotting. FIGS. 8C-8D: BALB/c mice were intradermally immunized with 10 μg ovalbumin (OVA) alone or in the presence of 3 FH VLPs or FljB. Immunization was repeated 3 weeks later. Serum anti-OVA antibody titer was measured 3 weeks after prime (FIG. 8C) and boost (FIG. 8D). Two-way ANOVA with Bonferroni's post-test was used to compare differences between groups at each protein concentration in FIG. 8A. One-way ANOVA with Tukey's multiple comparison test was used to compare differences between groups in FIGS. 8C and 8D (n=5 in both). *: p<0.05; ***: p<0.001. NS: not significant.

FIG. 8E show data that indicate FH VLPs boost ovalbumin (OVA) immunization. C57BL/6 mice were intradermally immunized with OVA alone or in the presence of 3, 10, or 30 μg FH VLPs. Serum anti-OVA antibody titer was measured 3 weeks later. Total IgG antibody titer was shown on the left and subtype IgG1 and IgG2c antibody titers were shown in the middle and right, respectively. n=4. One-way ANOVA with Dunnett's Multiple Comparison Test was used to compare differences between FljB-HBc and no adjuvant groups. *: p<0.05.

FIGS. 9A-9D show data that indicate more efficient uptake of FH VLPs than FljB in BMDCs. FIG. 9A: BMDCs were incubated with AF555-labeled FH VLPs, FljB, and HBc VLPs at equal molar concentrations (70 nM) of respective proteins, equivalent to 5:2.5:1 fluorescence intensity, in 8-well chamber slides. LysoTracker and Hoechst 33342 were added 1.5 and 2.5 hours later. Cells were subjected to fluorescence confocal imaging 3 hours later. Scale: 50 μm. In FIGS. 9B-9D, BMDCs were incubated with AF555-labeled FH VLPs, FljB, and HBc VLPs as in FIG. 9A in 96-well plates, respectively. Cells were harvested 3 and 24 hours later, stained with fluorescence-conjugated anti-CD11c followed by flow cytometry analysis. Cells were first gated based on SSC and FSC and then based on CD11c expression. Representative dot plots of percentage of AF555⁺ dendritic cells (DCs) at 3 hours were shown in FIG. 9B. Percentage of AF555⁺ DCs at 3 and 20 hours was shown in FIGS. 9C and 9D, respectively. n=5. One-way ANOVA with Tukey's multiple comparison test was used to compare differences between groups. *: p<0.05; ***: p<0.001. Experiments were repeated twice with similar results.

FIGS. 10A-10G present data showing FH VLPs stimulate dendritic cell (DC) maturation in vitro. BMDCs were incubated with FljB, FLA-ST, HBc VLPs, and FH VLPs in triplicate at equal molar concentrations (70 nM). Cells were harvested 20 hours later and stained with fluorescence-conjugated anti-CD11c, CD86, CD80, CD40 antibodies followed by flow cytometry analysis. Cells were first gated based on SSC and FSC and then based on CD11c expression. CD11c′ cells were analyzed for expression of CD40, CD80, and CD86. FIG. 10A: Representative histogram of CD40 expression and percentage of CD40^{hi} DCs. FIGS. 10B-10D: MFI of CD40 (FIG. 10B), CD80 (FIG. 10C), and CD86 (FIG. 10D) in CD11e DCs. FIGS. 10E-G: Percentage of CD40^{hi} (FIG. 10E), CD80^{hi} (FIG. 10F), and CD86^{hi} (FIG. 10G) in CD11c⁺ DCs. One-way ANOVA with Bonferroni's multiple comparison test was used to compare differences between groups. *: p<0.05; **: p<0.01; ***: p<0.001. Experiments were repeated twice with similar results.

FIGS. 11A-11E present data showing FH VLPs stimulate DC maturation in vivo. Lateral back skin of C57BL/6 mice was intradermally injected with 5 μg FH VLPs, 3.7 μg FljB, and 1.2 μg HBc VLPs (equal moles) in 20 μl or an equal volume of PBS to serve as control. Skin was dissected 24 hours later. Single-cell suspensions were prepared and stained with fluorescence-conjugated anti-CD11c, CD40, CD80, and CD86 antibodies followed by flow cytometry analysis. Cells were first gated based on FSC and SSC and then based on CD11c expression. CD11e cells were analyzed for CD40, CD80, and CD86 expression. FIG. 11A: Representative dot plots showing percentage of CD40^{hi} DCs. FIG. 11B: Comparison of percentage of CD40′ DCs among different groups. FIGS. 11C-E: MFI of CD40 (FIG. 11C), CD80 (FIG. 11D), and CD86 (FIG. 11E) in CD11e DCs. n=4. One-way ANOVA with Bonferroni's multiple comparison test was used to compare differences between groups. *: p<0.05; NS: not significant.

FIGS. 12A-12E show data that indicate FH VLPs induce more potent anti-FljB antibody response than FljB itself. WT and MyD88 KO mice were intradermally immunized with 5 μg FH VLPs or 3.7 μg FljB (equal moles). Immunization was repeated 3 weeks later. Serum antibody titer was measured 3 weeks after boost. FIG. 12A: Serum anti-FljB IgG antibody titer after FH VLP and FljB immunization. FIG. 12B: Serum anti-FljB IgG1 and IgG2c antibody titer after FljB immunization. FIG. 12C: Serum anti-FljB IgG1 and IgG2c antibody titer after FH VLP immunization. FIG. 12D: Serum anti-HBc IgG titer after FH VLP and FljB immunization. n=6. One-way ANOVA with Bonferroni's multiple comparison test was used to compare differences between groups. NS: not significant. FIG. 12E: Supernatant IL-12 levels in BMDC culture. BMDCs were incubated with FljB, FLA-ST, HBc VLP, and FH VLP in triplicate at equal molar concentrations. Supernatant IL-12 levels were analyzed by commercial ELISA kit at 20 hours. One-way ANOVA with Bonferroni's multiple comparison test was used to compare differences between groups. *, p<0.05; **, p<0.01; ***, P<0.001.

FIGS. 13A-13G show data that indicate significantly improved systemic safety of FH VLPs. Serum IL-6 levels were measured 6 (FIG. 13A) and 24 hours (FIG. 13B) after boost immunization of WT and MyD88 KO mice in FIG. 12. Serum IL-6 levels before immunization was also measured (baseline). BALB/c mice were intradermally immunized with 5 FH VLPs or 3.7 μg FljB (equal moles). Immunization was repeated 3 weeks later. Serum IL-6 levels were measured just before boost immunization or 6 (FIG. 13C) and 24 hours (FIG. 13D) after boost immunization. FIGS. 13E-13G: C57BL/6 mice were intradermally immunized with 20 μg FH VLPs or 14.8 μg FljB (equal moles). Immunization was repeated weekly for 3 weeks. Rectal temperature was measured right before each immunization (0 hour) or 24 hours after each immunization. n=6 in FIGS. 13A-13D and n=4 in FIGS. 13E-13G. *, p<0.05; **, p<0.01; ***, P<0.001. NS: not significant.

FIGS. 14A and 14B show data that indicate lack of overt toxicity from FH VLPs. FIG. 14A: Body weight of C57BL/6 mice in FIGS. 13E-13G was measured before immunization and one week after each immunization and compared among groups. FIG. 14B: Mice were sacrificed one week after the last immunization. Major organs, like heart, liver, spleen, lung, and kidney, were harvested, fixed in formalin, and then subjected to paraffin sectioning and H & E staining. Representative H & E-stained histological images were shown. Scale: 200 μm.

FIGS. 15A-15I show data that indicate high immunogenicity from FH VLPs for the nicotine vaccine example. BALB/c mice were intradermally immunized with KLH-Nic, FH-Nic, or FH-Nic in the presence of CpG, Alum, or CpG/Alum adjuvant for total 3 times with a 3-week interval. Serum NicAb titer was measured 3 weeks after the last immunization. Mice were then intravenously challenged with 0.03 mg/kg nicotine within one week after the last blood collection to measure serum NicAb titer. Five minutes later, brain and serum samples were isolated for quantification of tissue nicotine levels. FIGS. 15A-15D: Total IgG titer was shown in FIG. 15A and subtype IgG1 and IgG2a antibody titer was shown in FIGS. 15B & 5C, respectively. Ratio of IgG2a to IgG1 antibody titer was shown in FIG. 15D. FIGS. 15E and 15F show the brain and serum nicotine levels of different groups, respectively. Data of all groups were pooled for linear correlation analysis between NicAb titer and brain nicotine levels (FIG. 15G), brain and serum nicotine levels (FIG. 15H), and NicAb titer and serum nicotine levels (FIG. 15I). n=6-8 per group. *: p<0.05; **: p<0.01; ***: p<0.001.

FIGS. 16A-16C illustrate the design of FH-M2e and FH-OVA, two embodiments of the invention. FIG. 16A schematically illustrates a “recombinant model” based on the FH VLP disclosed herein as a vaccine platform. FIG. 16B: M2e sequences of human H1/H3 (SEQ ID NO:19), swine H1 (SEQ ID NO:20), Avian H5 (SEQ ID NO:21) and H7 (SEQ ID NO:22) influenza A viruses. Sequence differences of M2e in swine and avian viruses were bold highlighted. Schematic illustration of preparation of M2e-displaying FH VLPs (shorthanded as “FH-M2e”) as well as OVA-displaying FH VLPs (shorthanded as “FH-OVA”) is shown in FIG. 16C. Gene constructs were designed to replace D3 domain of FljB in FljB-HBc with 4 tandem copies of M2e or OVA₂₄₇₋₂₇₄ followed by E. Coli expression, purification, and self-assembly into VLPs.

FIGS. 16D-16H illustrate the confirmation of successful expression of FH-M2e and FH-OVA, two embodiments of the invention. In FIG. 16D, FH-M2e samples were subjected to SDS-PAGE analysis alongside reference samples where M2e is recombinantly expressed in HBc (“HBc-M2e) or FljB (”FljB-M2e″). In FIG. 16E, M2e-containing immunogens were subjected to Western Blotting analysis. Immune sera isolated from KLH-M2e-immunized mice were used to detect M2e-containing immunogens. In FIG. 16F, reference polypeptides HBc-OVA and FljB-OVA samples were subjected to SDS-PAGE analysis. In FIG. 16G, FH-OVA sample was subjected to SDS-PAGE analysis. Protein length and theoretical molecular weight of different immunogens were listed in FIG. 16H.

FIGS. 17A and 17B illustrate results from TEM and DLS analyses of FH-M2e VLPs. FIG. 17A: Refolded FH-M2e and HBc-M2e samples were subjected to TEM imaging and representative images were shown. Arrows (left panel) point to VLPs. Scale: 50 nm. FIG. 17B: FH-M2e samples were subjected to DLS analysis in Malvern Nanosizer. VLP distribution was shown on the left and VLP size and PDI were shown on the right.

FIGS. 17C-17E illustrate data on BMDC maturation induced by various immunogens expressing M2e. BMDCs were incubated with mixture of M2e peptides, HBc-M2e, FljB-M2e, and FH-M2e VLPs in triplicate at equal molar concentrations of 70 nM. Cells were harvested 20 hours later and stained with fluorescence-conjugated anti-CD11c, CD86, CD80, CD40 antibodies followed by flow cytometry analysis. Cells were first gated based on SSC and FSC and then based on CD11c expression. CD11c⁺ cells were analyzed for expression of CD40, CD80, and CD86. FIG. 17C: Representative histogram of CD40, CD80, CD86 expression and percentage of CD40hi, CD80hi, CD86hi DCs. FIG. 17D: MFI of CD40, CD80, and CD86 and percentage of CD40hi, CD80hi, CD86hi in CD11c+ DCs. One-way ANOVA with Bonferroni's multiple comparison test was used to compare differences between groups.*: p<0.05; ***: p<0.001. FIG. 17E summarizes results of DC uptake and maturation ability of diverse M2e immunogens.

FIGS. 18A-18C show data that indicate immunogenicity and protective efficacy of FljB-M2e. BALB/c mice were intramuscularly immunized with 5 μg FljB-m2e, 2.7m HBc-M2e that contained an equal amount of M2e, or a mixture of four different M2e peptides (10m each, synthesized by Thermo Fisher Scientific) in the presence of Alum adjuvant (Alhydrogel, Invivogen), or immunized with PBS as a negative control. Immunization was repeated 10 days later. FIG. 18A: Serum anti-M2e antibody titer of mice 10 days after boost by coating plates with M2e peptide mixtures. One-way ANOVA with Tukey's post-test was used to compare differences among groups. N.D.: not detected. NS: not significant. B-C. Mice were intranasally challenged with 4×LD50 of PR8 (H1N1) influenza virus 14 days after boost. Body weight (FIG. 18B) and survival (FIG. 18C) were monitored daily for 14 days. Mice were euthanized if body weight drop exceeded 25%. Body weight curve was not included after day 9 in M2e/Alum and HBc-M2e groups since only one mouse survived the challenge. n=4-5.

FIGS. 19A-19D show data that indicate FljB-M2e induces systemic cytokine release. Serum IL-6 (FIGS. 19A & 19B) and TNFα levels (FIGS. 19C & 19D) were measured before immunization and 3 and 18 hours after immunization. Two-way ANOVA with Tukey's multiple-comparison test was used to compare cytokine level differences at different time points within the same group. n=4-5. **, p<0.01; ***, p<0.001; NS: not significant.

FIGS. 20A-20C show data that indicate immunogenicity and protective efficacy of FH-M2e. BALB/c mice were intramuscularly immunized with 24.3 μg FH-m2e or 18.5 μg FljB-M2e in the presence or absence of 2 μg CpG 1018 (TriLink Biotechnologies) or PBS as a negative control. Immunization was repeated 10 days later. FIG. 20A: Serum anti-M2e antibody titer 10 days after boost. One-way ANOVA with Tukey's post-test was used to compare differences among groups. FIGS. 20B and 20C: Mice were intranasally challenged with 8×LD50 of PR8 (H1N1) influenza virus 14 days after boost. Body weight (FIG. 20B) and survival (FIG. 20C) were monitored daily for 14 days. Mice were euthanized if body weight drop exceeded 25%. n=5.

FIGS. 21A-21D show that FljB-M2e but not FH-M2e induces systemic cytokine release. Serum IL-6 (FIGS. 21A & 21B) and TNFα levels (FIGS. 21C & 21D) were measured before immunization and 3 and 18 hours after immunization. Two-way ANOVA with Tukey's multiple-comparison test was used to compare cytokine level differences at different time points within the same group. n=5. *, p<0.05; ***, p<0.001; NS: not significant.

FIGS. 22A and 22B show that FljB-M2e but not FH-M2e increases the body temperature, i.e., causes fever in mice. BALB/c mice were intradermally immunized with 25 μg FljB-M2e (FIG. 22A) or 33 μg of FH-M2e (FIG. 22B) that contained an equal amount of M2e. Rectal temperature of mice was measured before immunization and 24 hours after immunization. Student's t-test was used to compare differences before and 24 hours after immunization within the same group. n=4. *, p<0.05; NS: not significant.

FIGS. 23A-23C illustrate the data on cross-protective immunity from immunogens tested including the VLPs of the invention. BALB/c mice were intramuscularly immunized with 6.5 μg FH-M2e VLPs or 5 μg FljB-M2e that contained the same amount of M2e or immunized with PBS as a negative control. Immunization was repeated 3 weeks later. FIG. 23A: Mice were then challenged with 8×LD₅₀ of PR8 (A/Puerto Rico/8/34 H1N1) 4 weeks after boost. FIGS. 23B & C: BALB/c mice were challenged with 2×LD₅₀ of mouse-adapted influenza pandemic 2009 H1N1 (A/California/07/2009 H1N1) (FIG. 23B) or 5×LD₅₀ of A/Philippines/2/82 (H3N2) viruses (FIG. 23C) 4 weeks after boost. Body weight loss (upper) and survival (lower) were monitored daily for 14 days. Mice were euthanized if body weight drop exceeded 25%. n=5 in FIG. 23A; n=4 in FIGS. 23B & C. Log-rank test with Bonferroni correction was used to compare differences of survival between immunized and PBS groups in lower panels of FIGS. 23A-C. *, p<0.05; **, p<0.01.

FIGS. 24A-24F illustrate the data that suggest FH-OVA VLP induces more potent CTL production and confers more potent anti-tumor immunity. In FIGS. 24A and 24B, OT-I T cells were purified, stained with CFSE, and then intravenously injected into C57BL/6 mice. Mice were then intradermally injected with FH-OVA VLP, HBc-OVA VLP, FljB-OVA, OVA peptide that contained the same amount of OVA peptide, or PBS to serve as negative control. Draining LNs were collected 4 days later and single cells were prepared, stained with fluorescence conjugated anti-CD4 and CD8 antibodies, and then subjected to flow cytometry analysis. Representative dot plots showing CFSE+ cells in CD8+ T cells of different groups were shown in FIG. 24A. Statistical analysis of CFSE+ cells in CD8+ T cells was shown in FIG. 24B. In FIGS. 24C and 24D, C57BL/6 mice were subjected to FH-OVA VLP, HBc-OVA VLP, FljB-OVA, OVA peptide immunizations that contained the same amount of OVA peptide, or PBS to serve as negative control. Immunization was repeated two times with 2 weeks interval. Mice were then subcutaneously challenged with E.G7-OVA lymphoma cells. Tumor growth was monitored and shown in FIG. 24C. Survival of tumor-bearing mice was shown in FIG. 24D. In FIGS. 24E and 24F, C57BL/6 mice were similarly immunized as in FIGS. 24C and 24D followed by subcutaneous challenge of BL6F10-OVA melanoma cells. Tumor growth was monitored and shown in FIG. 24E. Survival of tumor-bearing mice was shown in FIG. 24F. n=4 in A-B; n=5 in C-D and E-F. One-way ANOVA with Tukey's post-test was used to compare differences among groups in A. Two-way ANOVA with Tukey's post-test was used to compare tumor volume differences at different time points between groups in C and E. Log-rank test with Bonferroni correction was used to compare differences of survival between groups in D and F. *, p<0.05; **, p<0.01; ***, p<0.001.

FIGS. 25A-25D illustrate data that suggest CpG 1018 further enhances anti-tumor efficacy of FH-OVA VLPs. FIG. 25A: OT-IT cells were adoptively transferred to C57BL/6 mice. Mice were intradermally immunized with FH-OVA VLPs in the presence or absence of CpG 1018 or PBS 24 hours later. Draining LNs were collected 4 days later, stimulated in vitro with CTL epitope of OVA in the presence of anti-CD28 antibody overnight. Brefeldin A was added and 5 hours later, cells were harvested, stained with fluorescence antibodies. Percentage of CFSE+ cells in CD8+ T cells and single, dual, and triple cytokine-secreting cells in CFSE+ CD8+ T cells were compared among groups. In FIGS. 25B-25D, mice were subjected to FH-OVA VLP immunization in the presence or absence of CpG 1018 or left non-immunized. Immunization was repeated 3 times with 2 weeks interval. Mice were then subcutaneously challenged with N₁₆F10-OVA melanoma 2 weeks after the last immunization. Tumor growth and survival were monitored for 30 days. Tumor growth rate, tumor development rate, and survival of tumor-bearing mice were shown in FIGS. 25B, 25C, and 25D, respectively. n=4 in A. n=5-6 in B-D. One-way ANOVA with Tukey's post-test was used to compare differences between groups in A. Two-way ANOVA with Tukey's post-test was used to compare tumor volume differences at different time points between groups in B. Log-rank test with Bonferroni correction was used to compare differences of survival between groups in D. *, p<0.05; **, p<0.01; ***, p<0.001.

FIGS. 26A and 26B illustrate data that show better systemic safety of FH-OVA VLP as compared to FljB-OVA. Serum IL-6 (FIG. 26A) and TNFα levels (FIG. 26B) were measured before immunization and 3 and 18 hours after the repeated immunizations of the different OVA immunogens described in Example 14 below. Two-way ANOVA with Tukey's multiple-comparison test was used to compare cytokine level differences at different time points within the group. n=5. *, p<0.05; ***, p<0.001; ****, p<0.0001. NS: not significant.

### DETAILED DESCRIPTION OF THE INVENTION

### I. DEFINITION

Unless otherwise noted, technical terms are used according to conventional usage. Definitions of common terms in molecular biology may be found, for example, in J. Krebs et al. (eds.), Lewin's Genes XII, published by Jones and Bartlett Learning, 2017 (ISBN 9781284104493); Robert A. Meyers (ed.), Molecular Biology and Biotechnology: a Comprehensive Desk Reference, published by Anmol Publications Pvt. Ltd, 2011 (ISBN 9788126531783); and other similar technical references.

As used in the specification and claims, the singular form “a”, “an”, or “the” includes plural references unless the context clearly dictates otherwise. For example, the term “a cell” includes a plurality of cells including mixtures thereof. It is further noted that the claims may be drafted to exclude any optional element. As such, this statement is intended to serve as support for the recitation in the claims of such exclusive terminology as “solely,” “only” and the like in connection with the recitation of claim elements, or use of a “negative” limitations, such as “wherein [a particular feature or element] is absent,” or “except for [a particular feature or element],” or “wherein [a particular feature or element] is not present (included, etc.) . . . ”.

When a dimensional measurement is given for a part herein, the value is, unless explicitly stated or clear from the context, meant to describe an average for a necessary portion of the part, i.e., an average for the portion of the part that is needed for the stated purpose. Any accessory or excessive portion is not meant to be included in the calculation of the value.

As used herein, the recitation of a numerical range for a variable is intended to convey that the invention may be practiced with the variable equal to any of the values within that range. Thus, for a variable which is inherently discrete, the variable can be equal to any integer value within the numerical range, including the endpoints of the range. Similarly, for a variable which is inherently continuous, the variable can be equal to any real value within the numerical range, including the endpoints of the range. As an example, and without limitation, a variable which is described as having values between 0 and 2 can take the values 0, 1 or 2 if the variable is inherently discrete, and can take the values 0.0, 0.1, 0.01, 0.001, or any other real values >0 and <2 if the variable is inherently continuous.

As used herein, “about” means within plus or minus 10%. For example, “about 1” means “0.9 to 1.1”, “about 2%” means “1.8% to 2.2%”, “about 2% to 3%” means “1.8% to 3.3%”, and “about 3% to about 4%” means “2.7% to 4.4%.”

As used here, the term “adjuvant” refers to a substance or a combination of substances that enhances an immune response to an antigen. Typically, an adjuvant is formulated as part of a vaccine to enhance the vaccine's ability to induce protection in the body against infection by a pathogen, e.g., a virus or bacterium. Adjuvants are considered to enhance the immune response to an antigen in the vaccine in at least one of the following ways: prolong the presence of antigen in the body (blood and/or tissue); help antigen-presenting cells absorb antigen; activate antigen-presenting cells, macrophages, and lymphocytes; and support the production of cytokines. Adjuvants are also used in the production of antibodies from immunized animals. An “adjuvant effect” refers to enhancement in the immune response to a selected antigen in a host that receives the vaccine.

As used herein the terms “administration,” “administering,” or the like, when used in the context of providing a pharmaceutical composition to a subject generally refers to providing to the subject one or more pharmaceutical compositions comprising the agent, e.g., the novel pharmaceutical compositions of the invention, by any means such that the administered compound achieves one or more of the intended biological effects for which the compound was administered. By way of non-limiting example, a composition may be administered parenteral, subcutaneous, intravenous, intracoronary, rectal, intramuscular, intra-peritoneal, transdermal, or buccal routes of delivery.

The term “antigen” as used herein refers to substance, e.g., an amino acid sequence, that is intended or designed to elicit a specific immunological response in a host— however, not all antigens successfully do so; those that do can be called “immunogen.” An “antigen,” as used herein, includes a full-length sequence of a protein, analogs thereof, or immunogenic portions thereof. The term “immunogenic portion” refers to a portion or fragment of a protein that includes one or more epitopes and thus elicits an immunological response. Such portions can be identified using any number of epitope mapping techniques, well known in the art. See, e.g., Epitope Mapping Protocols in Methods in Molecular Biology, Vol. 66 (Glenn E. Morris. Ed., Humana Press 1996, Totowa, N.J.). For example, linear epitopes may be determined by, e.g., concurrently synthesizing large numbers of peptides on solid supports, the peptides corresponding to portions of the protein molecule, and reacting the peptides with antibodies while the peptides are still attached to the supports. Such techniques are known in the art and described in, e.g., U.S. Pat. No. 4,708,871; Geysen et al., Proc. Natl. Acad. Sci. USA 1984, 81:3998-4002; Geysen et al., Molec. Immunol. 1986, 23:709-715. Similarly, conformational epitopes are readily identified by determining spatial conformation of amino acids such as by, e.g., X-ray crystallography and 2-dimensional nuclear magnetic resonance. Synthetic antigens are also included within the definition, for example, polyepitopes, flanking epitopes, and other recombinant or synthetically derived antigens. See, e.g., Bergmann et al., Eur. J. Immunol. 1993, 23:2777-2781; Bergmann et al., J. Immunol. 1996, 157:3242-3249; Suhrbier, A., Immunol, and Cell Biol. 1997, 75:402-408. The term “antigenic” as used herein means associated with an antigen or having characteristics of an antigen.

The term “high density” as used herein refers to a VLP structure or assembly displaying a protein (e.g., flagellin) or a portion thereof at a concentration that is significantly higher than reported in prior art for any VLP. In various embodiments, a HBc-based VLP structure formed by fusion proteins of the invention displays the flagellin protein or a portion thereof at a density at about 80% (weight/weight) as opposed to only 1-8% (weight/weight) of the total protein displayed in prior art examples. Viewed in another way, an embodiment of a “high density” display of flagellin or a portion thereof means there is no fewer than 100, 150, 180, or even 240 units in a single VLP structure.

As used herein, the terms “nucleic acid,” “polynucleotide,” and “oligonucleotide” are used interchangeably throughout and include DNA molecules (e.g., cDNA or genomic DNA), RNA molecules (e.g., mRNA), analogs of the DNA or RNA generated using nucleotide analogs (e.g., peptide nucleic acids and non-naturally occurring nucleotide analogs), and hybrids thereof. The nucleic acid molecule can be single-stranded or double-stranded. The double-stranded nucleic acid may have the two strands chemically linked and/or form at least one double-stranded region under suitable annealing conditions. The double-stranded region may contain at least one gap, nick, bulge, and/or bubble. In one embodiment, the nucleic acid molecules of the invention comprise a contiguous open reading frame encoding a fusion protein, an antibody, or a fragment, derivative, mutant, or variant thereof.

Nucleic acids encoding any of the various polypeptides disclosed herein may be synthesized chemically. Codon usage may be selected so as to improve expression in a cell. Such codon usage will depend on the cell type selected. Specialized codon usage patterns have been developed for E. coli and other bacteria, as well as mammalian cells, plant cells, yeast cells and insect cells. See for example: Mayfield et al., Proc. Natl. Acad. Sci. USA. 2003 100(2):438-42; Sinclair et al. Protein Expr. Purif. 2002 (1):96-105; Connell N D. Curr. Opin. Biotechnol. 2001 12(5):446-9; Makrides et al. Microbiol. Rev. 1996 60(3):512-38; and Sharp et al. Yeast. 1991 7(7):657-78.

General techniques for nucleic acid manipulation are described for example in Sambrook et al., Molecular Cloning: A Laboratory Manual, Vols. 1-3, Cold Spring Harbor Laboratory Press, 2 ed., 1989, or F. Ausubel et al., Current Protocols in Molecular Biology (Green Publishing and Wiley-Interscience: New York, 1987) and periodic updates, herein incorporated by reference. The DNA encoding the polypeptide is operably linked to suitable transcriptional or translational regulatory elements derived from mammalian, viral, or insect genes. Such regulatory elements include a transcriptional promoter, an optional operator sequence to control transcription, a sequence encoding suitable mRNA ribosomal binding sites, and sequences that control the termination of transcription and translation. The ability to replicate in a host, usually conferred by an origin of replication, and a selection gene to facilitate recognition of transformants is additionally incorporated.

The recombinant DNA can also include any type of protein tag sequence that may be useful for purifying the protein. Examples of protein tags include but are not limited to a histidine tag, a FLAG tag, a myc tag, an HA tag, or a GST tag. Appropriate cloning and expression vectors for use with bacterial, fungal, yeast, and mammalian cellular hosts can be found in Cloning Vectors: A Laboratory Manual, (Elsevier, N.Y., 1985).

The expression construct is introduced into the host cell using a method appropriate to the host cell. A variety of methods for introducing nucleic acids into host cells are known in the art, including, but not limited to, electroporation; transfection employing calcium chloride, rubidium chloride, calcium phosphate, DEAE-dextran, or other substances; microprojectile bombardment; lipofection; and infection (where the vector is an infectious agent). Suitable host cells include prokaryotes, yeast, mammalian cells, bacterial cells, or plants.

Proteins disclosed herein can also be produced using cell-translation systems. For such purposes the nucleic acids encoding the polypeptide must be modified to allow in vitro transcription to produce mRNA and to allow cell-free translation of the mRNA in the particular cell-free system being utilized (eukaryotic such as a mammalian or yeast cell-free translation system or prokaryotic such as a bacterial cell-free translation system.

The expression “pharmaceutically acceptable carrier, additive or excipient” is intended to include a formulation, or substance used to stabilize, solubilize and otherwise be mixed with active ingredients to be administered to living animals, including humans. This includes any and all solvents, liquid or solid filler, diluent, excipient, encapsulating material, dispersion media, coatings, antibacterial and antifungal agents, isotonic and absorption delaying agents, and the like, compatible with pharmaceutical administration. Except insofar as any conventional media or agent is incompatible with the active compound, such use in the compositions is contemplated. Each carrier must be “acceptable” in the sense of being compatible with the other ingredients of the formulation and not injurious to a human subject. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Wetting agents, emulsifiers and lubricants, such as sodium lauryl sulfate, magnesium stearate, and polyethylene oxide-polypropylene oxide copolymer as well as coloring agents, release agents, coating agents, sweetening, flavoring and perfuming agents, preservatives and antioxidants can also be present in the compositions.

As used herein, the term “recombinantly linked,” when referring to amino acid sequences, peptides and polypeptides, means that the amino acid sequences, peptides and polypeptides are connected to each other as a result of original genetic materials (DNA or RNA) encoding them having been recombined often after being broken up first. For example, “recombinantly linked” amino acid sequence A and sequence B can be: “-A-B-,” “-A₁-B-A₂-,” or “-A₁-B₁-A₂-B₂-,” “-A₁-B₁-A₂-N₂-A_{3,}” and so on with or without linker sequences anywhere in the chain, where A₁, A₂, A₃, and so on are portions of the original sequence A, and B₁, N₂, N₃, and so on are portions of the original sequence B. Amino acid sequences are recombinantly linked as long as they can be produced as part of a fusion protein that is encoded by genetic materials that have been manipulated using recombinant techniques. Recombinantly linked amino acid sequences can be produced using host cells, cell translation systems or otherwise chemically synthesized.

Similarly, as used herein, the term “recombinantly inserted,” when referring to two amino acid sequences, means that a first sequence is inserted into a second one as a result of the original genetic material for the first one being inserted into the original genetic material for the second one through recombinant techniques before both amino acid sequences are expressed as part of a fusion protein. Once the design of the new protein sequence is completed where the first amino sequence is “recombinantly inserted” into the second, the new sequence can be produced using host cells, cell translation systems, plants, or otherwise chemically synthesized.

As used herein, the term “subject” refers to any animal (e.g., a mammal), including, but not limited to humans, non-human primates, rodents, canines, and the like, which is to be the recipient of a particular treatment. Typically, the terms “subject” and “patient” are used interchangeably herein in reference to a human subject.

As used herein, the term “substantial portion” refers to part of an entity, short of its entirety, but with sufficient portions thereof such that it can function in a fashion that is equivalent to the full entity as far as this invention is concerned. For example, in the context of the flagellin domain(s), a substantial portion of a domain or domains can generate equivalent levels of immunity as the full domain or domains. In certain embodiments, such substantial portion refers to at least 90 or 95 percent of the full amino acid sequence of the full domain or domains. In the context of a VLP-forming polypeptide, a substantial portion thereof refers to a portion of the full-length polypeptide that, once expressed, can still form a VLP in an aqueous environment. For example, the first 149 aa of the full HBc sequence would qualify as a substantial portion of the HBc sequence in the context of being used as a VLP-forming polypeptide. Determination of exactly how much of the full sequence is needed in order to qualify as a substantial portion can be performed through routine investigation by one of ordinary skills in the proteomics or vaccine-research art using standard procedures such as point deletion or mutagenesis combined with functionality assessment.

The term “vaccine” as used herein refers to a biological preparation that induces immunity to a particular disease. A vaccine typically contains an antigen that resembles a disease-causing microorganism or compound and is often made from weakened or killed forms of the microbe, its subunit(s), epitope-containing portion(s), its toxins or one of its surface proteins, as well as genetic materials such as RNA that encodes parts of the microbe. Vaccines can be prophylactic (e.g., to prevent or ameliorate effects of a future infection by a pathogen such as the influenza virus), or therapeutic (e.g., vaccines against certain cancers that are being developed).

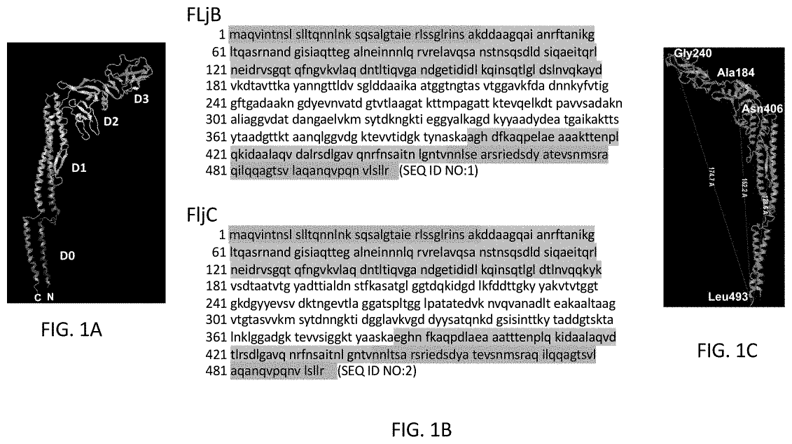

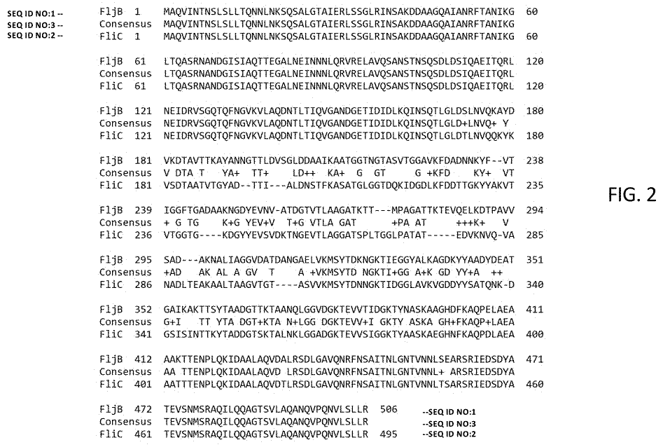

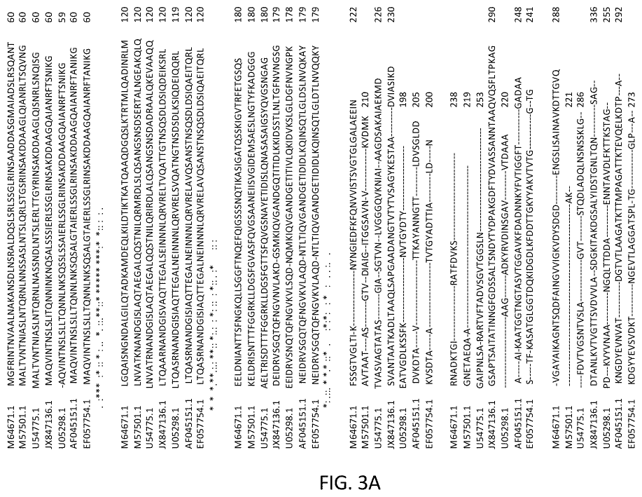

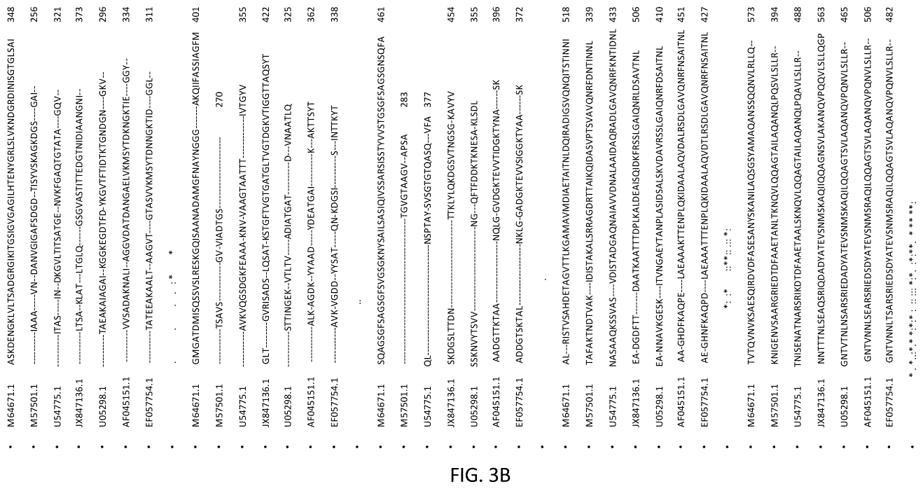

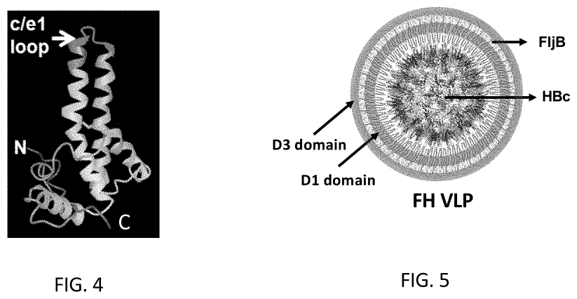

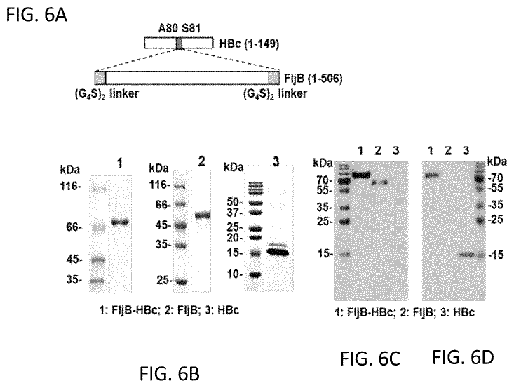

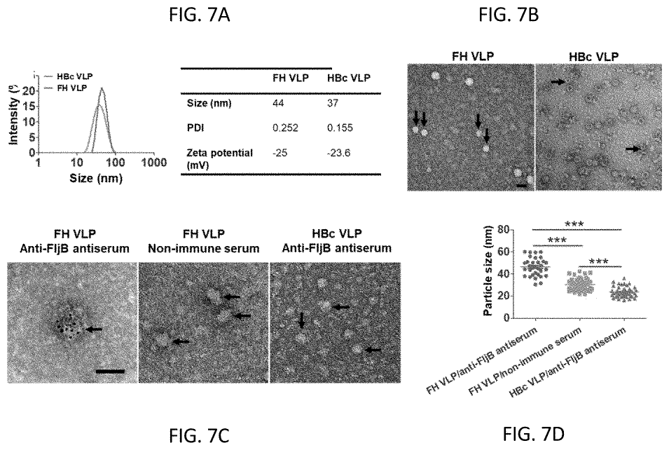

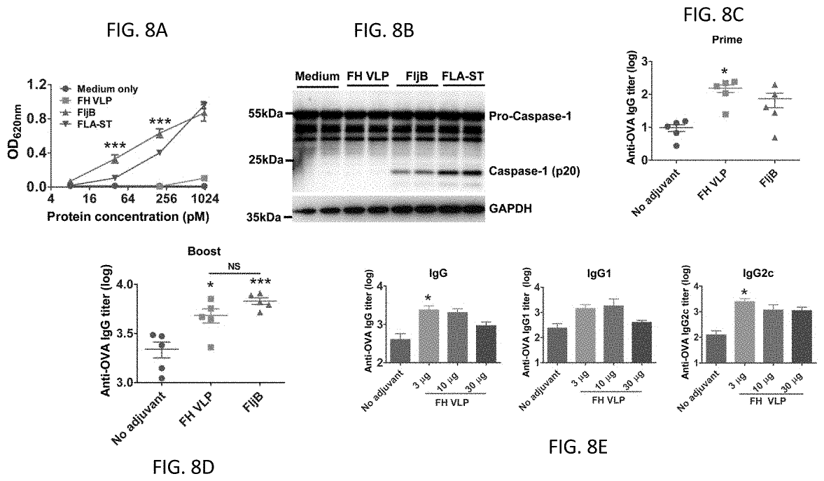

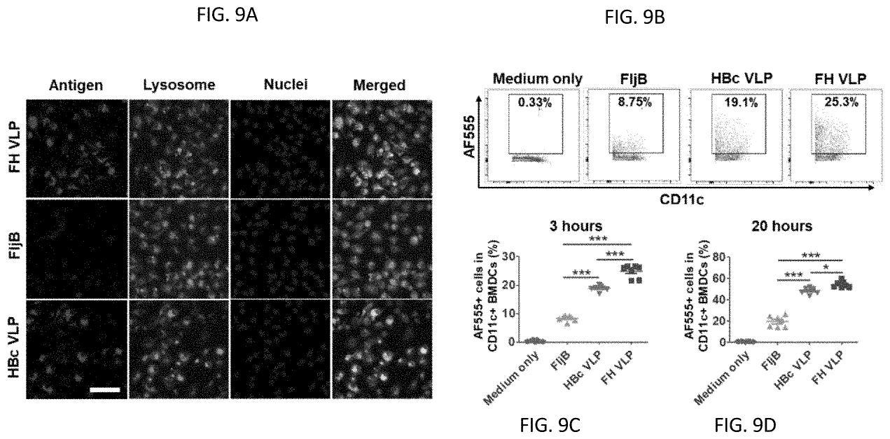

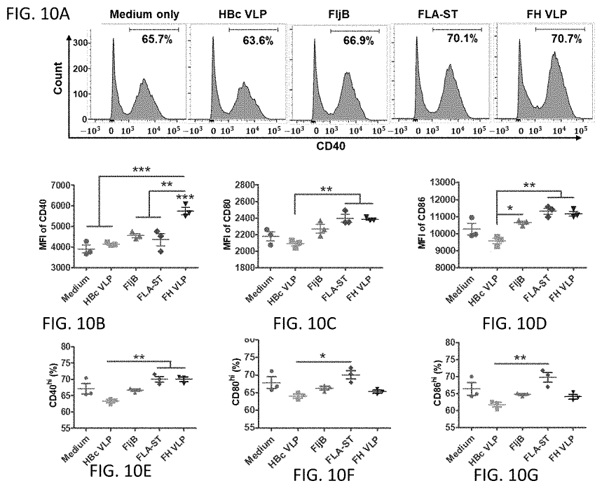

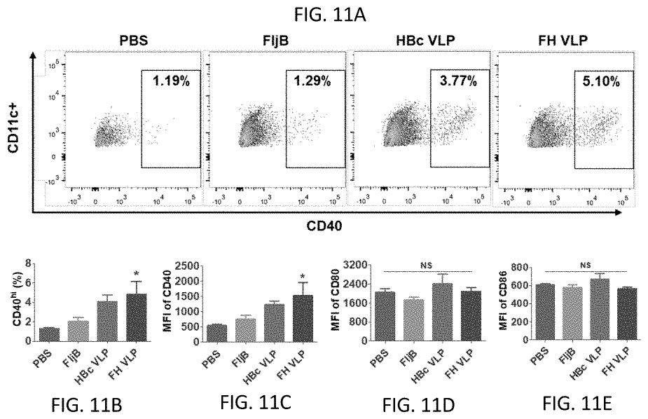

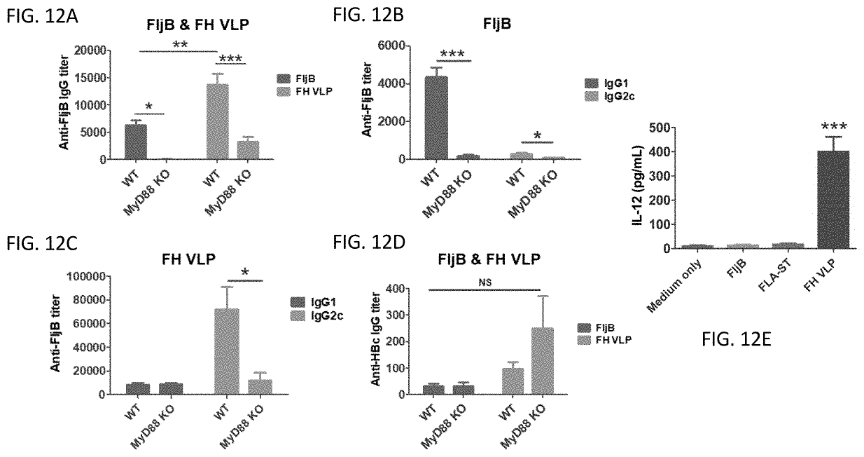

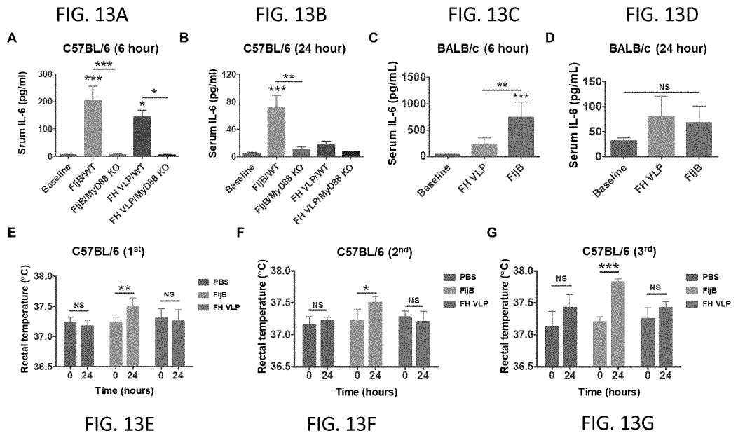

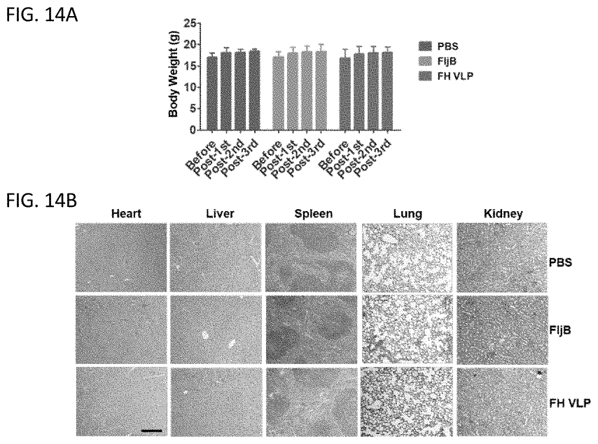

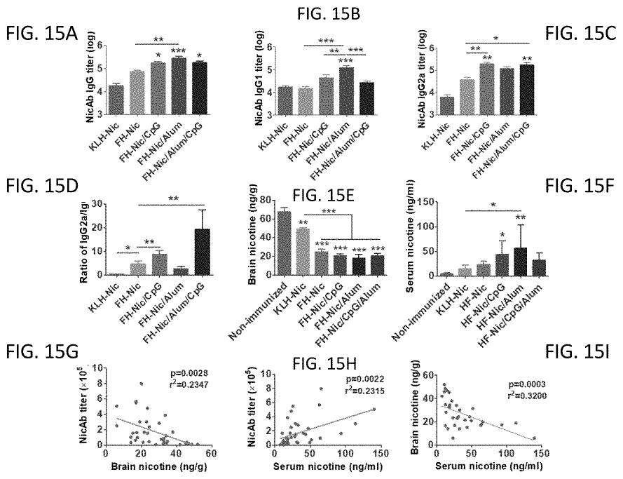

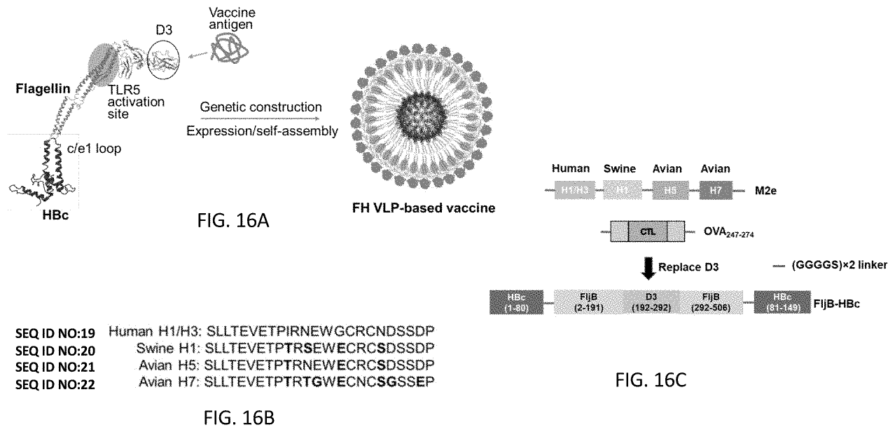

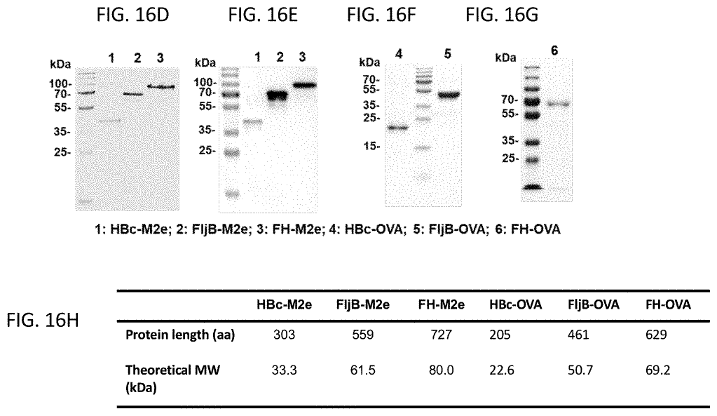

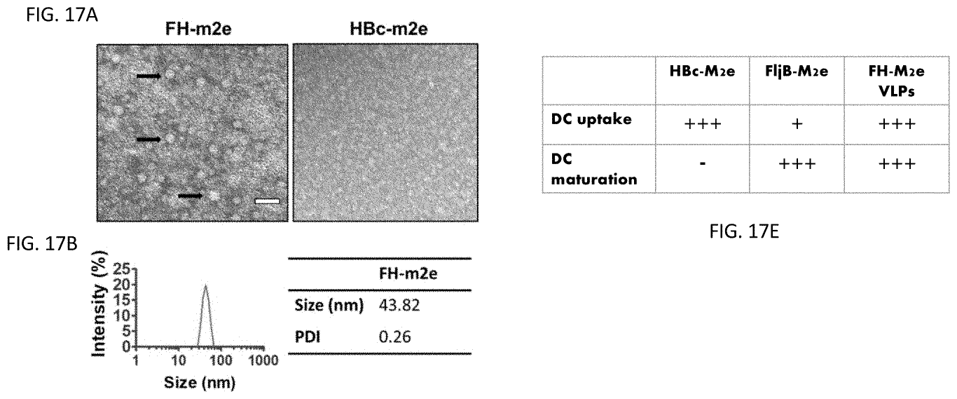

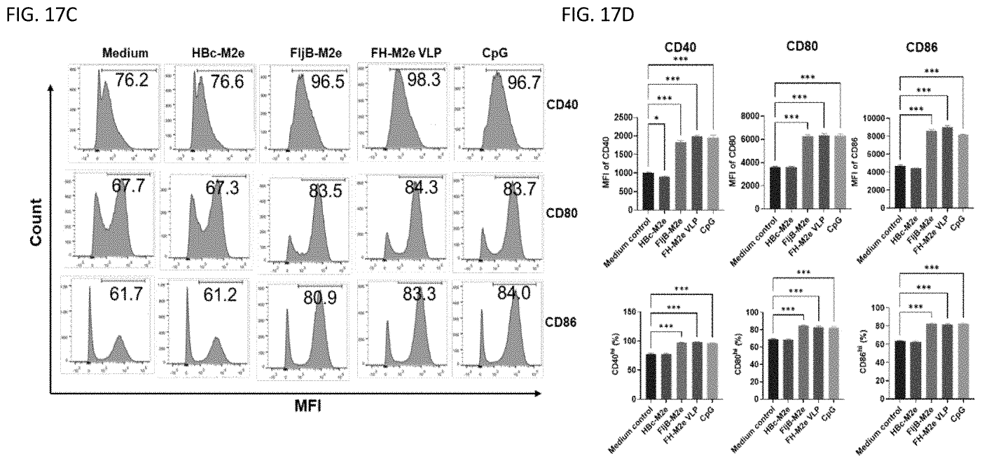

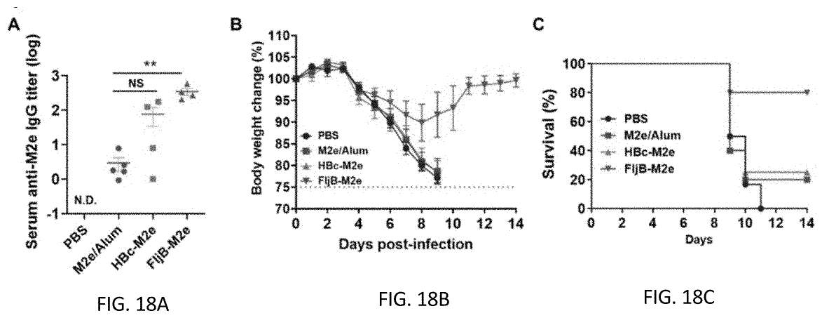

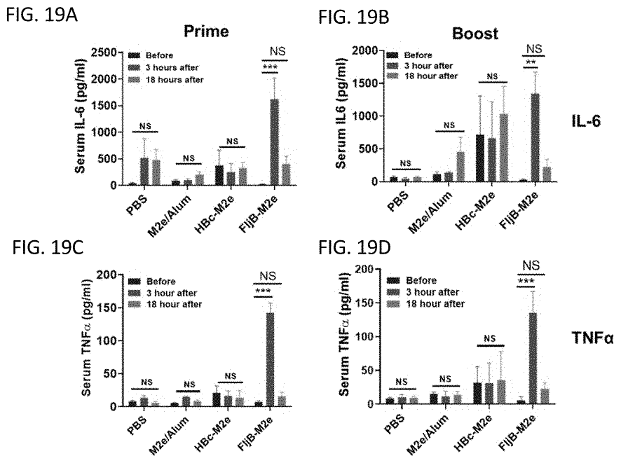

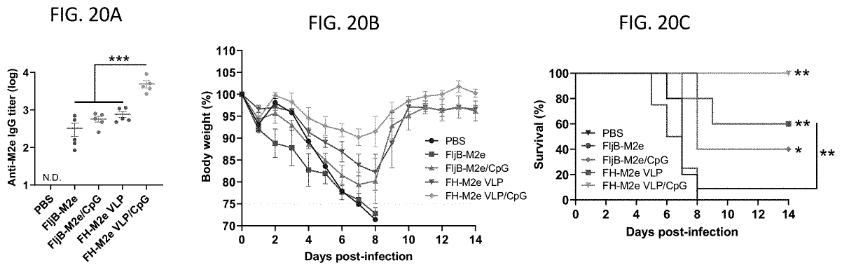

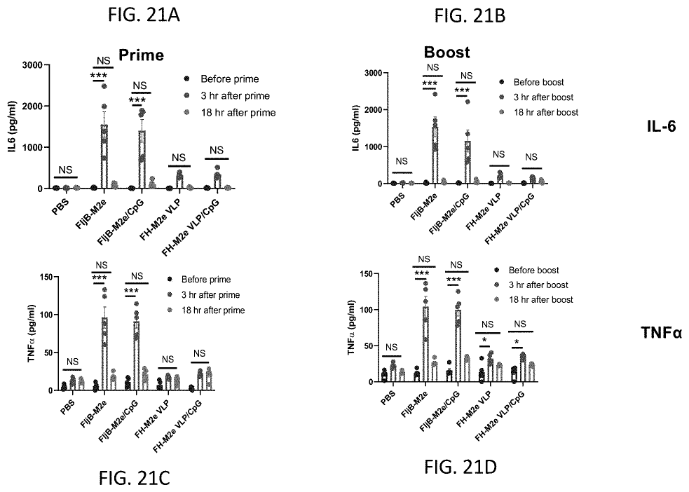

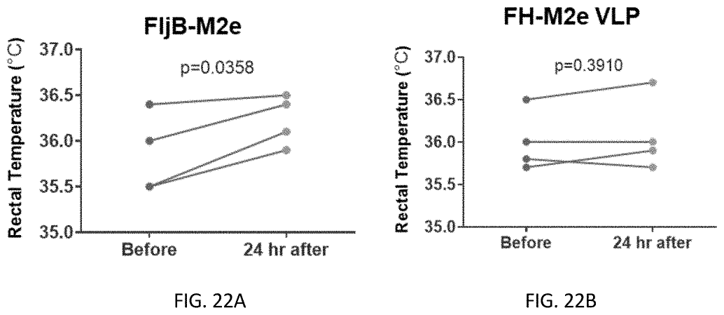

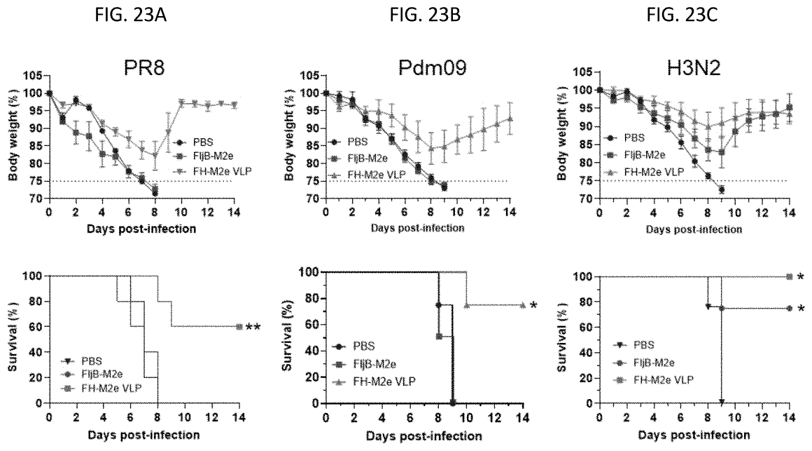

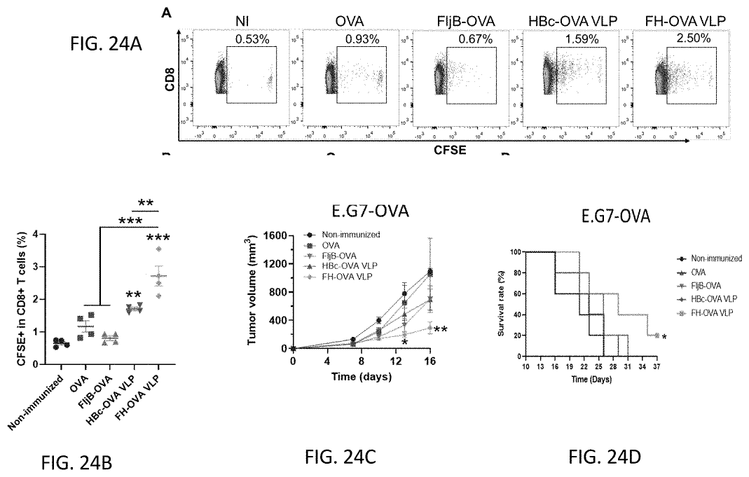

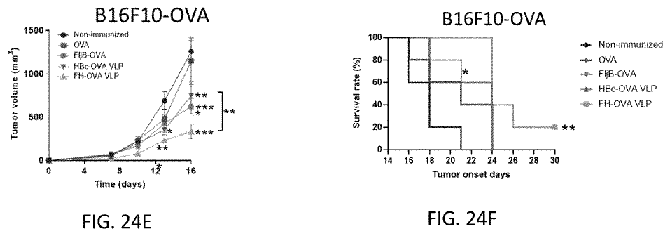

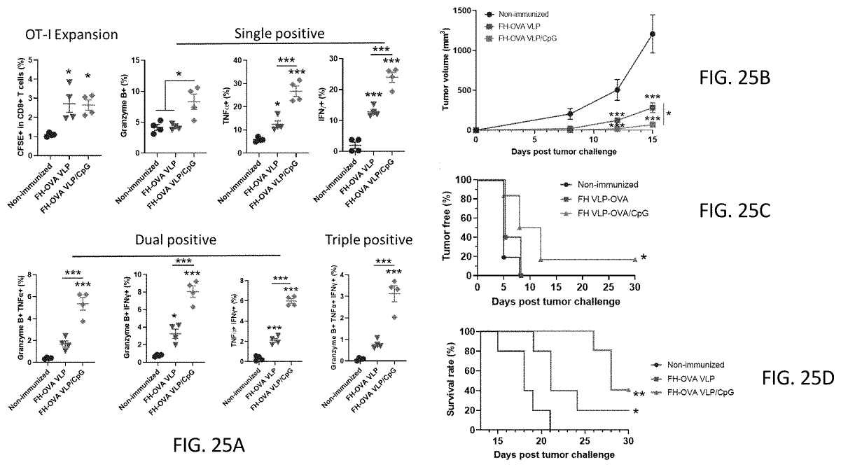

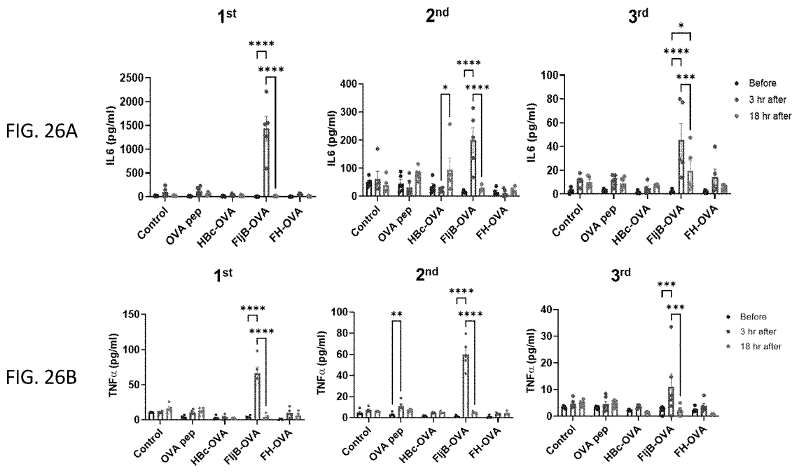

The flagellin protein is found in flagellated bacteria including but are not limited to Salmonella (e.g., Salmonella enterica and Salmonella bongori), Escherichia coli, Vibrio vulnificus, Campylobacter coli, Bacillus subtilis, Burkholderia pseudomallei and Pseudomonas aeruginosa. In Salmonella enterica serovar Typhimurium (S. Typhimurium), for instance, while there are two different forms of flagellin, FljB and FliC, only one is expressed at any given time in the cell (J. R. McQuiston et al., J. Clin. Microbiol. 2004, 42(5): 1923-32). FIG. 1A shows the crystal structure of flagellin, starting from its N-terminus in blue to its C-terminus in red, in sequence of the rainbow colors. As shown, the N- and C-termini of flagellin are closely juxtaposed, with the D0 domain being closest to the inner core when assembled in the bacteria to form the flagellum filament. Therefore, the D0 domain, defined in terms of the folded 3D structure, includes the two most far-apart segments (SEQ ID NOs:4 and 5, respectively from N′ to C′) in the protein sequence that occupy the N- and C-termini respectively (FIG. 1B, highlighted in turquoise blue). Next, the D1 domain occupies two neighboring segments (SEQ ID NOs:6 and 7, respectively from N′ to C′) that, in actual folding, come close to each other (FIG. 1B, highlighted in gray). As an illustrating example, the demarcation of domains in the FliC protein from S. Typhimurium strain LT2, are from amino acid positions counting from the N-terminus as follows: 1-42 (D0), 43-176 (D1), 176-397 (D2, D3), 398-455 (D1), and 456-495 (D0). And as shown in FIG. 2, the D0 and D1 domains between FljB and FliC are highly conserved.

This homology is further confirmed by looking across at seven different variants of flagellin proteins across different bacterial species (FIGS. 3A and 3B): while the flagellin proteins show greatest variations in the mid-section (D2 and D3 domains), they are highly conserved in regions close to both the N- and C-termini where the D0 and D1 domains are situated.

VLP-forming polypeptides can self-assemble into virus-like structures, but because these structures contain no viral genetic material, they are not infectious to the host. Typically, VLP-forming polypeptides are structural proteins found in a virion, such as the capsid. Polypeptides or structural proteins from many virus families have been found to be capable of forming VLPs, including Flaviviridae (e.g., Hepatitis B and C viruses), Retroviridae (e.g., HIV), Parvoviridae (e.g., adeno-associated virus), Paramyxoviridae (e.g., Nipha) and bacteriophages (see, A. Zeltins, Mol. Biotechnol. 2013, 53 (1): 92-107). VLPs, due to their small sizes and strong immunogenicity that results from high density display of viral surface proteins, may be useful as vaccines. An example of a VLP-forming polypeptide is the Hepatitis B core protein (HBc), with its crystal structure shown in FIG. 4.

According to the present invention, a flagellin protein or a portion thereof is recombinantly expressed as operably linked to a VLP-forming polypeptide; the resultant fusion proteins then, in turn, self-assemble into their own VLP structure or assembly displaying, in high density (e.g., no fewer than 100, 150, 180, or even 240 units in a single VLP structure), the flagellin protein or a portion thereof. In various embodiments, about 180 or 240 molecules each having a flagellin protein or a portion thereof have been found in a single VLP of the invention, which provides it at much higher concentrations than any prior art has been able to. Further, the flagellin protein or a portion thereof displayed by the VLP is oriented in a way such that the D0 and D1 domains are not exposed on the surface of the VLP (or otherwise easily accessible from outside the VLP) whereas the D3 domain and possibly a part of the D2 domain are (FIG. 5). The novel VLP assembly of the invention based on the fusion proteins of the invention has turned out to be a versatile, highly effective and safe immunogenic platform and can be used as at least part of a vaccine or vaccine carrier.

In prior art, flagellin has been chemically conjugated to HBc VLPs (Y. Lu, et al., Biotechnol. Bioeng. 2013, 110 (8): 2073-85; Y. Lu, et al., Sci. Rep. 2016, 6: 18379). In those studies, only a small fraction of HBc molecules were conjugated with flagellin, and low-density display of flagellin on HBc VLP surface was found not to significantly impact its TLRS activation ability (ditto). Besides HBc VLPs, flagellin was also displayed on influenza Matrix 1 (M1) VLP surface by co-expression of membrane-anchored flagellin and M1 in insect cells (B. Z. Wang, et al., J. Virol. 2008, 82 (23): 11813-23; E.J. Ko, et al., Vaccine 2019, 37 (26): 3426-3434). The content of flagellin in M1 VLPs was found to be about 1.3% or 8% of total protein (weight/weight). Low-density display of flagellin on M1 VLPs showed comparable TLRS activation ability to free flagellin (B.Z. Wang, et al., supra).

In sharp contrast to these prior strategies that resulted in low-density display of flagellin on VLP surface, self-assembly of the novel fusion protein where flagellin is expressed recombinantly linked to a VLP-forming polypeptide, led to high-density display of flagellin onto HBc VLP surface with —80% flagellin content in the VLP assembly. High-density display of flagellin on HBc VLP surface also showed at least 100-fold reduced TLR5 activation ability as compared to free flagellin. Besides significantly reduced TLR5 activation ability, the VLPs of the invention were also shown to have lost most of their NLRC4 inflammasome and Caspase 1 activation ability. D0/D1 domains of flagellin, crucial for TLR5 and NLRC4 inflammasome activation, are likely buried in the interior of the VLPs of the invention without access to TLR5 or NLRC4 inflammasome.

Despite significant reduction of TLR5 activation ability, surprisingly, VLPs of the invention showed similar adjuvant effects in boosting co-administered ovalbumin (OVA) immunization when compared to free flagellin. OVA is a standard reference protein for vaccination research. As the major protein constituent of chicken egg whites, ovalbumin is sufficiently large and complex to be mildly immunogenic. As a result, it is widely used as an antigen for immunization research and validation. Inventor's surprise finding means adjuvant effects of flagellin could be maintained even with a significant loss of TLR5 activation ability. Also, VLPs of the invention induced more than two-fold anti-flagellin antibody titer than flagellin itself, indicating improved immunogenicity. The high immunogenicity of the VLPs of the invention is supported by their increased uptake by APCs and induction of stronger dendritic cell (DC) maturation than free flagellin. The increased uptake of the VLPs of the invention may simply reflect more efficient uptake of particulate antigens by phagocytosis than soluble antigen uptake by non-receptor-mediated endocytosis (macropinocytosis), or the increased uptake of the VLPs of the invention may also have been mediated by cell surface receptors. The exact reasons for the surprise observation of the greater immunogenicity from the VLPs of the invention are to be determined by further investigation.

In a preferred embodiment of the invention, a full-length flagellin protein from Salmonella, FljB (506 aa, SEQ ID NO:1), or a portion thereof, is recombinantly inserted into the sequence of a VLP-forming polypeptide, the Hepatitis B core (HBc) protein or a substantial portion thereof, preferably anywhere within its c/el loop, or by replacing part or all of the c/el loop (FIG. 4). The resultant fusion protein, as well as other embodiments using other flagellin variants or portions thereof, is shorthanded as “FljB-HBc” or “FH” and their VLP assembly as “FH VLP” in this disclosure. In one embodiment, the full-length HBc protein sequence (183 aa, adw subtype, Genbank Accession No. AET99094.1, SEQ ID NO:17) is incorporated into the new fusion protein. In an alternate embodiment, the VLP-forming polypeptide utilized to make the fusion protein is a portion of the HBc protein sequence (e.g., about the first 149 aa of the full HBc, i.e., SEQ ID NO:16, as the remainder of the protein sequence proves to be unnecessary for self-assembly), or another suitable viral structural protein, such as those forming part of a viral capsid, or a portion thereof. Whether a full-length HBc or a portion thereof is used, the VLP formed by congregates of many such proteins or polypeptides is shorthanded as “HBc VLP” in this disclosure. Examples of other candidate VLP-forming polypeptides that may be employed to practice the invention include those well known in the art, and are not limited to: HBc variants (e.g., the woodchuck hepatitis B virus core protein (WHBc)); hepatitis B virus S antigen (HBs); human papillomavirus (HPV) proteins; bovine papillomavirus (BPV) proteins; human immunodeficiency virus (HIV) proteins; simian immunodeficiency virus HIV chimera (SHIV) proteins; duck hepatitis B virus (DHBV) proteins; and hepatitis E virus (HEV) proteins (see, e.g., E. Grgacic et al., Methods 2006, 40: 60-65).

Inventors' work with VLPs made in accordance with principles of the invention, as exemplified by the FH VLPs, showed significantly reduced ability in activating TLR5 or inducing systemic IL-6 release than the flagellin protein itself. Flagellin but not immunization by VLPs of the invention was found to significantly increase rectal temperature of mice (a sign of fever), indicating improved safety profile with VLPs of the invention. Moreover, VLPs of the invention showed similar adjuvant effects to flagellin in boosting a co-administered antigen (e.g., OVA) immunization and VLPs of the invention induced more than two-fold higher anti-flagellin antibody titer than flagellin itself. Consistent with improved immunogenicity, VLPs of the invention could be more efficiently taken up by bone marrow-derived dendritic cells (BMDCs) and stimulate more potent dendritic cell (DC) maturation than flagellin. In addition, it was found that VLPs of the invention were a more immunogenic carrier than the original flagellin, VLP-forming polypeptides and the VLPs they assemble into, as well as other benchmarks used in the vaccine industry: (1) FH VLPs, an embodiment of the VLPs of the invention, performed better in all immunogenic tests than HBc VLPs, and the widely used keyhole limpet hemocyanin (KLH) for vaccine development, when FH VLPs were chemically conjugated to an nicotine hapten molecule; (2) VLPs formed by an exemplary fusion protein of the invention (FH) that is further recombinantly fused to an epitope (e.g., M2e in an influenza virus, or a CTL epitope of the ovalbumin protein, which are 146 and 48 aa including linkers) by displacing the highly variable D3 domain of FljB (FIG. 16A), were found to successfully form VLP and display said epitope in high density and elicit higher levels of immunity than flagellin and HBc VLPs without unwanted side effects. In a further example of the invention, hemagglutinin antigen 1 (HA₁) of pandemic influenza 2009 H1N1 virus (over 300 aa) is successfully displayed on FH VLP surface by replacement of D3 domain of FljB. Data presented herein support FH VLPs as a more versatile delivery platform than HBc VLPs (see, e.g., data and description in connection with FIG. 17A) for vaccine development.

While not wishing to be bound by any particular theory, our data suggest that improvements in both safety profile and immunogenicity seen in the novel vaccine platform provided by the present invention in comparison to existing flagellin-based vaccine candidates may be contributed to the following: (a) the novel VLP assembly of the invention successfully hides the D0 and D1 domains or significant portions thereof inside the assembly--sequences essential for the recognition by TLR5 have been located in those two domains (e.g., ammino acid sequence segments 1-99 and 427-455 in S. Typhimurium FljB and segments 1-99 and 416-434 in S. Typhimurium FliC)--preventing access to them, thereby reducing unwanted TLR5 activation and associated systemic adverse reactions such as cytokine storm and fever; (b) immunogenicity improves due to flagellin or flagellin-associated immunogenic agent being transitioned from a soluble form to a particulate form; and (c) immunogenicity improves due to high-density display and concentration of flagellin or flagellin-associated immunogenic agent, as the flexibility of flagellin's D3 domain (and to a less extent, the D2 domain) allows the insertion of or substitution by not just one or more epitopes of small or moderate sizes but antigens of much larger sizes--the VLP-forming polypeptides, such as HBc, are not capable of such a feat without interfering or preventing the proper VLP assembly. In other words, the present invention has succeeded in combining features from both flagellin and prior art VLPs in an unexpected way that somehow remedies each other's shortcomings.

Application-wise, the fusion proteins of the invention and the resultant VLPs can be useful in the vaccine field in at least the following ways: (a) as adjuvant in a therapy co-administered with another antigenic agent, e.g., any type of vaccine including but not limited to: recombinant, conjugated, VLP-based, or toxoid-based vaccines; (b) conjugated to an immunogen, whether protein-based, polysaccharide-based or hapten-based; (c) further recombinantly fused to an immunogenic sequence, whether a single or multiple epitopes, a full-length antigen or a portion thereof (FIG. 16A).

First using nicotine vaccine as a model, the inventors compared relative immunogenicity of the VLPs of the invention to flagellin, HBc VLPs, and widely used KLH for purpose of nicotine vaccine development. VLPs of the invention (as exemplified by FH VLPs) were found to be a more immunogenic carrier than the widely used KLH, and KLH was more immunogenic than flagellin and HBc VLPs when tested for nicotine vaccine development. Immunization by VLPs of the invention chemically conjugated to nicotine induced about 4 times higher NicAb titer and more significantly inhibited nicotine entry into the brain than immunization by KLH-nicotine conjugates. It was further found that incorporation of Alum or CpG adjuvant into immunization by a vaccine based on VLPs of the invention that are conjugated to nicotine could further increase antibody production against nicotine and reduce nicotine entry into the brain after nicotine challenge.

In addition to high immunogenicity, VLPs of the invention showed a good systemic safety profile. Clinical adverse reactions include a significant increase of serum c-reactive protein (CRP) levels, significant increase of serum IL-6 levels, and increase of body temperature in some participants. Such adverse reactions were markedly absent from tested conducted here. For instance, VLPs of the invention induced more transient systemic IL-6 release than free flagellin in C57BL/6 mice and VLPs of the invention failed to significantly increase serum IL-6 levels in BALB/c mice. Furthermore, VLPs of the invention did not increase body temperature in mice even after repeated administrations at relatively high doses, while soluble flagellin at an equivalent dose consistently and significantly increased the body temperature of mice. No significant microscopic organ damage or body weight change relative to control group was observed either following immunization by VLPs of the invention. These results support the finding that VLPs of the invention have shown improved systemic safety when compared to flagellin, and can is indeed a good candidate for vaccines.

Besides developing vaccines using the presently disclosed VLPs in a “conjugate model,” the inventors then discovered through “recombinant models” (FIG. 16A) where antigenic epitope(s) or an immunogenic portion of an antigen can be recombinantly inserted into the flexible D3, or D2 domain of flagellin for high-density display on the surface of a fusion-protein-formed VLP according to principles of the invention. Even full-length proteins may be inserted into one of the flagellin domains, or may be used to replace D3 and/or D2 domain for high-density display on the surface of the VLPs of the invention. In various embodiments, an immunogenic sequence larger than about 50, 100, 200, 300, 400, 500, 600, 700, 800, 900, or 1000 amino acid residues is inserted into the fusion protein of the invention, with or without replacing a portion of the sequence derived from flagellin. Thus, VLPs of the invention provides a more versatile delivery platform than HBc VLPs considering c/el loop of HBc only allows insertion of relatively short antigenic epitopes while the D3 and D2 domains of flagellin allow insertion of foreign antigens with much less restriction on lengths or 3D structures.

This disclosure provides working examples where an infectious disease vaccine model and a cancer vaccine model are tested using recombinant fusion proteins of the invention. In most of the examples, the diverse antigens also replaced D3 domain of flagellin to prepare flagellin-based vaccines to compare its immunogenicity and safety with FH VLP-based vaccines; or inserted into c/el loop of HBc to explore its impact on HBc VLP assembly and prepare HBc or HBc VLP-based vaccines to compare its immunogenicity with FH VLP-based vaccines.

In a first recombinant model, a universal influenza vaccine was designed where ectodomains of influenza matrix protein 2 (M2e) from four different variants were recombinantly inserted to replace the D3 domain of the flagellin and expressed as part of a protein fused to a portion of HBc to assemble into a VLP structure (shorthanded as “FH-M2e” in this disclosure). With linkers, the antigenic sequence inserted into FH VLP was 146 amino acids residues in length. This fusion-protein-based vaccine embodiment was found to confer cross-protective immunity against various influenza A viral strains, offering strong potential as a universal influenza vaccine that can elicit anti-M2e antibody responses against seasonal, pandemic, and pre-pandemic viruses.

In a second recombinant model, the CTL epitope of ovalbumin (OVA) was also designed to replace the highly variable D3 domain of flagellin for display on the FH VLP surface (shorthanded as “FH-OVA” in this disclosure). Once expressed, our data suggest FH-OVA can be used to elicit OVA-specific CTL responses against OVA-expressing E.G7 lymphoma or Bl6F10 melanoma, therefore is a potential vaccine against multiple types of cancer.

FH VLP-based vaccines according to principles of the invention showed higher immunogenicity and protective efficacy as compared to FljB or HBc VLP-based vaccines. FH-M2e VLP mainly induces anti-M2e antibody responses to confer protection against viral challenges, while FH-OVA VLP mainly induces CTL responses to protect against OVA-expressing lymphoma or melanoma challenges. The ability of the FH VLP platform to elicit potent humoral and cellular immune responses against surface-displayed antigens supports its broad application in vaccine development against extracellular or intracellular pathogens or tumors. The high immunogenicity of FH VLP platform is expected to be due to the adjuvant effects of the particular form of FljB, which was found to potentiate Thl-biased antibody responses and vaccine-specific CTL responses. In comparison, soluble FljB was found to mainly potentiate Th2-biased antibody responses and only weakly stimulate vaccine-specific CTL responses.

Inventors further found that immunogenicity and protective efficacy of FH VLP-based vaccines could be substantially increased by incorporation of certain adjuvants, such as a clinical CpG 1018 adjuvant. To their surprise, a relatively low dose of CpG 1018 (2 μg) was found to significantly increase FH-M2e VLP-induced anti-M2e antibody titer by 6.5 folds and increase FH-M2e VLP-induced protection from 60% to 100%. Interestingly, 2 μg CpG 1018 was ineffective to enhance FH-OVA VLP-induced CTL responses and anti-tumor immunity (data not shown), which was achieved with increased CpG 1018 dose at 40 pg. This might reflect the differential CpG 1018 dose required to potentiate humoral and cellular immune responses elicited by FH VLP-based vaccines. Furthermore, inventors found incorporation of 2 μg CpG 1018 into FH-M2e VLP immunization didn't increase the risk of systemic adverse reactions in mice. Significantly increased immunogenicity and preserved safety strongly support low dose CpG 1018-adjuvanted FH-M2e VLP-based universal influenza vaccine approach in both human and animal models. As 2 μg CpG 1018 in mice translates to about 400 μg CpG 1018 in a regimen administered to human, in a preferred embodiment of low-dose administration, about or less than 400 μg per dose of CpG 1018 is administered as an adjuvant with a vaccine based on a VLP disclosed herein with. Incorporation of 40 μg CpG 1018 into FH-OVA VLP immunization induced a low level of IL-6 and TNFα release in mice, which gradually reduced after repeated immunizations, similar to that observed in FljB-OVA immunizations. Examples disclosed herein used OVA as a model to test whether compositions of the invention could effectively present tumor antigens, but it is within the contemplation of the present invention that tumor-associated antigens (TAAs) or neoantigens can be readily displayed on FH VLP surface by replacing D3 domain of FljB (or otherwise inserting into a flagellin sequence) to elicit potent Thl-biased antibody and CTL responses and incorporation of CpG 1018 can be further increase FH VLP-based tumor vaccine efficacy.

Similar to what was found with the conjugate model, tests conducted with the recombinant model also found that FH VLP-based vaccines showed significantly reduced ability to cause body temperature increases in mice or to induce systemic cytokine release, supporting the good safety profile of FH VLP platform. The good systemic safety of FH VLP or FH VLP-based vaccines is likely due to its significantly reduced TLR5 activation ability considering TLR5 activation leads to IL-6 synthesis, which further activates CRP release. The highly oriented surface display of FljB likely embeds its D1 domain, responsible for TLR5 activation, in interior of FH VLPs.

Accordingly, the present invention further provides a pharmaceutical composition, or a kit containing the fusion proteins of the invention preferably assembled as VLPs. The composition or kit can further include a pharmaceutically acceptable carrier, additive or excipient. The fusion proteins of the invention, as exemplified by the FljB-HBc fusion protein, can be conveniently expressed in bacterial systems such as E. Coli, which the pharmaceutical industry has lots of experience with, in large scale and refolded in vitro to form VLPs. Alternately, there are many other ways to produce the VLPs of the invention, for example, direct purification under native condition after expression in E. Coli. The fusion protein of the present invention can also be expressed by any other expression system known in the art, including systems that use yeast, other bacteria, insect cells, mammalian cells, cell-free expression systems, plants, or transgenic animals followed by purification under native or denatured conditions.

In a preferred embodiment, E. Coli cells are first used to express the fusion protein of the invention. After E. Coli cells are lysed, the harvested supernatant is purified under denatured condition. Subsequently, the fusion proteins are allowed to self-assembly into VLP after undergoing dialysis to remove denaturing agent (Urea). The process is well practiced and can find a good description in “Protein production and purification” (Nat. Methods 2008, 5(2): 135-146.), “Overview of the Purification of Recombinant Proteins” (Curr. Protoc. Protein Sci. 2015, 80: 6.1.1-6.1.35.), or “Protein Expression Handbook” (accessible online at thermofisher.com/content/dam/LifeTech/global/Forms/PDF/protein-expression-handbook.pdf).

The superior immunogenicity, good safety, and potential for large-scale production support the use of the VLPs of the invention as vaccine carriers, vaccines, or adjuvant.

In various embodiments, the fusion proteins and VLPs of the present invention can be utilized to make vaccines against all kinds of pathogens and diseases, including and are not limited to: influenza viruses, Hepatitis viruses, human papillomavirus (HPV), human immunodeficiency viruses (HIV), the Norwalk virus, Ebola, and Marburg viruses.

In one embodiment, a vaccine against severe acute respiratory syndrome coronavirus 2 (SARS-CoV-2), the virus causing COVID-19, is made based on principles of the present invention. Viral surface spike (S) protein of SARS-CoV-2 that mediates viral infection, or a portion thereof, is recombinantly expressed to be displayed on FH VLP surface to elicit neutralization antibodies in order to prevent viral infection. More conserved intracellular nucleocapsid (N) protein is also chosen to display on FH VLP surface to elicit cytotoxic T lymphocyte (CTL) responses with potentially broad cross-protective immunity to eliminate virus-infected cells and promote recovery. Thus, S and N co-displayed FH VLPs (SN/FH VLPs) may serve as a highly immunogenic and safe vaccine to tackle current and potential future COVID pandemics.

### EXAMPLES

### Example 1

### Construction of Recombinant FljB-HBc Plasmid

FljB gene of S. Typhimurium strain LT2 (SEQ ID NO:1) and partial HBc gene encoding 1-149 region of adw subtype (SEQ ID NO:16) were synthesized by Thermo Fisher Scientific. A forward primer containing an Nde I recognition site, his-tag sequence, and thrombin cleavage site and a reverse primer containing an Xho I recognition site and stop codon were used to amplify the FljB gene.

A forward primer containing an Nde I recognition site and a reverse primer containing an Xho I recognition site were used to amplify the HBc (1-149 aa) gene.

Polymerase chain reaction (PCR) products of FljB and HBc genes were then subjected to Xho I and Nde I digestion and subsequently ligated into pET-29a vector digested with the same enzymes.

Overlapping PCR was used to insert FljB gene into c/el loop (N75-L84 in the sequence) of HBc at A₈₀-S81. In fact, insertion can be made at any other location within the c/el loop such as P79-A₈₀, or a portion or even the entire c/el loop can be replaced with the sequence that is being inserted. A flexible linker, e.g., one with one or more glycines and serines, such as GGGGSGGGGS or (G4S)2 (SEQ ID NO:18) was inserted between HBc and FljB sequences to increase protein chain flexibility during refolding. Other well-known flexible linkers can also be inserted between HBc and FljB sequences to increase protein chain flexibility. PCR products were purified, digested with Xho I and Nde I, and ligated into pET-29a vector. Successful ligation was confirmed with sequencing after transformation of ligated pET-29a vector into competent DH5a cells.

### Example 2

### FljB-HBc self-assembles into VLPs

As shown in FIG. 4, the c/el loop of HBc is localized in the flexible region of two a-helices and form the tip of spikes on the HBc-VLP surface. As it turns out, insertion of FljB into c/el loop of HBc did not pose significant steric hindrance to self-assembly of the resulting FljB-HBc fusion protein into VLPs, thus allowing high-density display of FljB on HBc VLP surface, as depicted in FIG. 5.

In Example 1, DNA was constructed to express full-length FljB between A₈₀ and S81 of c/el loop of HBc and (G45)2 linker was inserted between FljB and HBc sequences to increase protein chain flexibility (FIG. 6A). Recombinant FljB-HBc, FljB, and HBc with a theoretical molecular weight of 71.3, 52.5, and 17.7 kDa, respectively, were expressed in E. coli, purified, refolded, and analyzed by SDS-PAGE (FIG. 6B). Western blotting analysis confirmed the presence of both FljB and HBc in FljB-HBc fusion protein. As shown in FIG. 6C, FljB-HBc and FljB but not HBc samples showed positive bands at the right position when PVDF membrane was blotted with anti-FljB antiserum. And as shown in FIG. 6D, FljB-HBc and HBc but not FljB samples showed positive bands at the right position when PVDF membrane was blotted with anti-HBc antiserum.

Next, FljB-HBc and HBc samples were subjected to dynamic light scattering (DLS) analysis in Zetasizer Nano ZS (Malvern). As shown in FIG. 7A, FH VLPs and HBc VLPs had a size of 44 and 37 nm for each structure, respectively, with a PDI value less than 0.3 for both particles. Zeta potential was also measured and found to be -25 mV for FH VLPs and −23.6 mV for HBc VLPs at neutral pH (FIG. 7A), in line with the theoretical isoelectric point (pI) of 5.03 and 5.73 for FljB-HBc and HBc, respectively (ExPASy). Slightly more net negative charge of FH VLPs can be explained by the slightly lower pI of FljB-HBc. TEM analysis found that both samples contained round-shaped particles less than 50 nm in diameter (FIG. 7B). It was further found that HBc VLPs and FH VLPs each had two particle sizes, consistent with report that HBc could self-assemble into particles with two icosahedral symmetries (T=3 and T=4) (P. T. Wingfield, et al., Biochemistry 1995, 34 (15): 4919-32). VLP particles where T=3 and T=4 contained 180 and 240 copies of HBc, respectively, with T =3 particles slightly smaller than T =4 particles (see ditto). In comparison to HBc VLPs, FH VLPs showed brighter signals following negative staining and TEM, which may have been caused by reduced scattering of electron beams due to dense FljB coating (see C. A. Scarff, et al., J. Vis. Exp. 2018, (132): e57199, doi:10.3791/57199).

In addition, immunogold labeling TEM was conducted to verify the presence of FljB on FH VLP surface. As shown in FIG. 7C, FH VLPs showed positive immunogold signals after staining with anti-FljB antiserum but not non-immune serum. As a negative control, HBc VLPs showed no immunogold signals after immunogold staining with anti-FljB antiserum (FIG. 7C). These results had clearly indicated the presence of FljB on HBc VLP surface.

Particle size of the different VLPs were further measured after immunogold labeling. As shown in FIG. 7D, FH VLPs had a size of 46 nm after immunogold staining with anti-FljB antiserum and a size of only 30 nm after immunogold staining with non-immune serum. Such a difference is likely caused by the immunogold staining that revealed FljB on FH VLP surface. Interestingly, the distance between Leu493 (end of D0 domain) and Ala184 (middle of the D2/D3 domain) of FliC was about 15 nm (FIG. 1C), which matched very well with the above size difference—in other words, the size difference of the FH VLPs from the two measurements are fully explained by the size of the FljB protein as it extends. HBc VLPs had a size of 24 nm after immunogold staining with anti-FljB antiserum (FIG. 7D). Immunogold labeling TEM revealed surface-displayed FljB and allowed the inventors to more accurately measure FH VLP size, which was much bigger than HBc VLPs (FIG. 7D).

### Example 3

### FH VLPs showed Impaired TLR5 and Caspase-1 Activation but Preserved Adjuvant Effects

Flagellin activates both TLR5 and NLRC4 inflammasome. The ability of FH VLPs to activate TLR5 and NLRC4 inflammasome was explored and compared with FljB and FLA-ST. FLA-ST is ultrapure FliC purified from S. Typhimurium based on manufacturer's description. HEK293 cells co-transfected with murine TLR5 and SEAP reporter gene were incubated with FH VLP, FljB, and FLA-ST at different molar concentrations of respective proteins. As shown in FIG. 8A, FH VLPs showed much weakened ability to activate TLR5 than FljB and FLA-ST. The lowest concentration of FljB, FLA-ST, and FH VLPs to significantly activate TLR5 was 8, 40, and 1000 pM, respectively (FIG. 8A), indicating 25-fold reduced TLR5 activation ability of FH VLPs as compared to FLA-ST and more than 100-fold reduced TLR5 activation ability of FH VLPs as compared to FljB.

Next, the ability of FH VLPs to activate NLRC4 inflammasome was tested. NLRC4 inflammasome is a multi-protein complex, the activation of which cleaves pro-Caspase-1 to form p10/p20 heterodimer. Here Caspase-1 activation was directly analyzed following incubation of BMDCs with FH VLPs, FljB, and FLA-ST to reflect their NLRC4 inflammasome activation ability. As shown in FIG. 8B, Caspase-1 p20 subunit could be readily detected after BMDC incubation with FljB and FLA-ST but not with FH VLPs. This result indicates significant loss of NLRC4 inflammasome activation ability in FH VLPs.

Considering that FljB does not require strong activation of innate immunity to exert its adjuvant effects, adjuvant effects from FH VLPs on boosting co-administered OVA immunization in BALB/c mice were tested. As shown in FIG. 8C, FH VLPs but not FljB significantly increased anti-OVA antibody titer after prime. FH VLPs also similarly increased anti-OVA antibody titer to FljB after boost (FIG. 8D).

To explore whether adjuvant effects of FH VLPs could be observed in another species of mice (C57BL/6 mice) and whether FH VLPs could dose-dependently increase OVA-induced antibody production, C57BL/6 mice were intradermally immunized with OVA alone or in the presence of increasing FH VLP doses (3, 10, and 30 μg). Results show that low dose but not medium or high dose of FH VLPs significantly increased anti-OVA antibody titer in C57BL/6 mice (FIG. 8E). Furthermore, low-dose FH VLPs more significantly increased anti-OVA IgG2c antibody titer than IgG1 antibody titer.

These mouse immunization studies indicate that FH VLPs possessed potent adjuvant effects across different species despite the lack of significant TLR5 and NLRC4 inflammasome activation abilities.

Example 4

### More efficient uptake by BMDCs of FH VLPs than FljB

Effective vaccine carriers need to be efficiently taken up by antigen presenting cells (APCs) in a mammalian subject. Accordingly, uptake of FH VLPs and FljB was tested in BMDCs. Soluble proteins are often endocytosed and sorted through early endosomes, late endosomes, and lysosomes (J. S. Blum, et al., Annu. Rev. Immunol. 2013, 31: 443-73). Particulate antigens are often phagocytosed and end up in phagolysosomes formed by fusion of phagosomes and lysosomes. Considering lysosomes and phagolysosomes have a pH of 4-4.5, LysoTracker capable of staining acidic organelles was used to stain both structures in this example.

BMDCs were incubated with AF555-conjugated FH VLPs, FljB, and HBc VLPs at equal molar concentrations of respective proteins. FH VLPs showed more significant uptake than FljB as evidenced by much stronger AF555 signals in FH VLP group than FljB group (Antigen, FIG. 9A). The majority of AF555 signals in both FH VLP and FljB groups were found to overlap with LysoTracker signals (Merged, FIG. 9A). HBc VLPs were also included for comparison and we found BMDCs could also significantly take up HBc VLPs into lysosomes (FIG. 9A).

BMDC uptake of FH VLPs, FljB, and HBc VLPs at equal molar concentrations was also examined through flow cytometry. As shown in FIGS. 9B and 9C, the percentages of AF555+ DCs were 25% in FH VLP group, 19% in HBc VLP group, 8% in FljB group, and less than 1% in a medium-only group. Percentage of AF555+ DCs was significantly higher in the FH VLP group than in the FljB group (FIG. 9C). Percentage of AF555⁺ DCs was also significantly higher in the FH VLP group than in the HBc VLP group (FIG. 9C). The same trend was also observed after 20 hours of incubation (FIG. 9D). The same experiment was repeated by incubation of BMDCs with equal fluorescence intensities of FH VLPs, FljB, and HBc VLPs, equivalent to about 1:2:5 molar concentrations of FljB-HBc, FljB, HBc. Again, significantly increased uptake of FH VLPs was observed as compared to soluble FljB although HBc VLPs showed the most significant uptake this time (data not shown). These data indicate that the VLPs of the invention, FH VLPs, could be more efficiently taken up by BMDCs in comparison to FljB.

Similar results were observed once FH has been recombinantly engineered to carry an antigenic sequence (e.g., M2e) and allowed to fold and assemble into FH VLPs that also display the antigen in high density.

### Example 5

### FH VLPs Stimulate more Potent DC Maturation than FljB both in vitro and in vivo

Besides efficient uptake, stimulation of DC maturation by vaccine carriers is also crucial to elicit potent vaccine-specific immune responses. Accordingly, the relative ability of FH VLPs to stimulate BMDC maturation is tested alongside FljB, FLA-ST, and HBc VLPs.

In brief, BMDCs were stimulated with FH VLPs, FljB, FLA-ST, and HBc VLPs at equal molar concentrations of respective proteins. Surface expression of costimulatory molecules CD40, CD80, and CD86 was tested 20 hours later. It was found that BMDCs could be divided into two populations based on CD40 expression: CD40^{high} (CD40^{high}) and CD40^{low (CD}40^{lo}), as shown in FIG. 10A. MFI of CD40, CD80, and CD86 was compared among groups, and MFI of CD40 was found to be significantly increased in the FH VLP group but not in FljB, FLA-ST, or HBc VLP group when compared to that in the medium-only group (FIG. 10B). MFI of CD40 in FH VLP group was also significantly higher than those in FljB, FLA-ST, and HBc VLP groups (FIG. 10B).

FH VLPs and FLA-ST slightly increased MFI of CD80 and CD86 (FIGS. 10C and 10D). WI of CD80 and CD86 in FH VLP and FLA-ST groups was significantly higher than in the HBc VLP group but not the medium-only group (FIGS. 10C and 10D). And MFI of CD86 in the FljB group was also significantly higher than that in the HBc VLP group (FIG. 10D).

Next, percentages of CD40^{hi}, CD80^{hi}, and CD86^{hi} DCs among the different groups were compared. The percentage of CD40^{hi}, CD80^{hi}, and CD86^{hi} DCs was found to be significantly higher in the FLA-ST group than in the HBc VLP group, and the percentage of CD40^{hi} DCs was significantly higher in the FH VLP group than in the HBc VLP group (FIGS. 10E-10G). The above results indicate that FH VLPs were the most potent stimulator to increase surface expression of costimulatory molecules, among which CD40 expression showed the most significant increase. Although FH VLPs could stimulate BMDC maturation, the ability of FH VLPs to stimulate BMDC maturation was much weaker than LPS (data not shown). The inability of FljB to significantly stimulate murine BMDC maturation was in line with previous report.

Further next, whether FH VLPs could also stimulate DC maturation in vivo was put to test. To explore this, C57BL/6 mice were intradermally injected with FH VLPs, FljB, and HBc VLPs. Skin was dissected 24 hours later for test of CD40, CD80, and CD86 expression on CD11c⁺ DCs. As shown in FIGS. 11A and 11B, percentage of CD40^{hi} cells in CD11c⁺ DCs was significantly increased in FH VLP but not in either the FljB or HBc VLP group when compared to that in the PBS group. MFI of CD40 was also significantly increased in FH VLP but not in either the FljB or HBc VLP group when compared to that in the PBS group (FIG. 11C). As in the in intro studies, no significant increases of MFI of CD80 or CD86 were found in FH VLPs, FljB, or HBc VLPs when compared to that in the PBS group (FIGS. 11D and 11E). These results indicate that FH VLPs could also significantly increase CD40 expression and stimulate DC maturation in vivo.

### Example 6

### FH VLPs are more Immunogenic than FljB in vivo

More efficient uptake and stimulation of DC maturation indicate that FH VLPs might have improved immunogenicity as compared to FljB. To prove this, mice were immunized with FH VLPs and FljB (equal moles) and anti-FljB antibody responses were then measured and compared between groups.

Flagellin was reported to mainly induce Thl-biased IgG1 antibody responses with high dependence on MyD88. To test FH VLP-induced antibody isotype and the dependence on MyD88, MyD88 knock-out (KO) mice were also included in the above immunization studies. As shown in FIG. 12A, FH VLPs induced 2.2-fold higher anti-FljB antibody titer than FljB. Lack of MyD88 almost completely abrogated FljB-induced antibody production but only partially inhibited FH VLP-induced anti-FljB antibody production (FIG. 12A).

FH VLP and FljB-induced isotype IgG1 and IgG2c antibody titers were also compared. As shown in FIG. 12B, FljB was found to induce strong IgG1 and weak IgG2c antibody titer, hinting the induction of Th2-biased antibody responses. Lack of MyD88 almost completely abrogated FljB-induced IgG1 and IgG2c antibody production (FIG. 12B). In comparison, FH VLPs induced strong anti-FljB IgG2c and weak IgG1 antibody titer (FIG. 12C), hinting the induction of Thl-biased antibody responses. Lack of MyD88 partially reduced FH VLP-induced IgG2c antibody production and showed no significant effects on FH VLP-induced IgG1 antibody production (FIG. 12C).

Due to the crucial role of IL-12 in skewing Thl -biased immune responses (C. Heufler, et al., Eur. J. Immunol. 1996, 26 (3): 659-68), IL-12 levels in BMDC stimulation studies were measured and it was found that FH VLPs but not FljB or FLA-ST stimulated significant IL-12 release (FIG. 12E). This result at least partially explained strong induction of Thl-biased IgG2c antibody responses by FH VLPs. Further, the inventors measured anti-HBc antibody titer and found that FH VLPs failed to induce significant anti-HBc antibody production (FIG. 12D).

The above studies strongly indicate that FH VLPs are more immunogenic than FljB in stimulating anti-FljB antibody production.

### Example 7

### Improved Systemic Safety of FH VLPs

Significantly reduced TLR5 activation ability implicates that FH VLPs might have improved safety as compared to FljB. To prove this, serum IL-6 levels were first measured following FH VLP and FljB immunization of WT and MyD88 KO mice in the above studies (FIGS. 12A-12D) since IL-6 had been linked to systemic adverse reactions of flagellin-based vaccines.

As shown in FIG. 13A, serum IL-6 levels were significantly increased in WT mice at 6 hours after both FH VLP and FljB immunizations. Serum IL-6 levels returned to baseline levels 24 hours after FH VLP immunization and remained at relatively high levels 24 hours after FljB immunization in WT mice (FIG. 13B). This result indicates that FH VLPs induce more transient systemic IL-6 release than FljB. Interestingly, serum IL-6 levels failed to significantly increase after FH VLP or FljB immunization in MyD88 KO mice (FIGS. 13A and 13B), hinting crucial roles of MyD88 in mediating FljB-induced systemic IL-6 release.

The relative ability of FH VLPs and FljB to induce systemic IL-6 release was also tested in BALB/c mice. FljB was found to significantly increase serum IL-6 levels at 6 hours, while FH VLPs failed to significantly increase serum IL-6 levels at the same time (FIG. 13C). Serum IL-6 levels reduced to baseline levels 24 hours after immunization in both groups (FIG. 13D). These results indicate that the ability to induce systemic IL-6 release is much more reduced in FH VLPs than in FljB.

Besides induction of systemic IL-6 release, flagellin-based vaccines had also induced systemic adverse reactions, like fever, in clinical trials. Next, the abilities of FH VLPs and FljB to each induce fever-related systemic adverse reactions in murine models were compared. To test this, C57BL/6 mice were intradermally immunized with increased doses of FH VLPs and FljB (20 and 14.8 μg, respectively) weekly for 3 weeks and rectal temperature was measured right before and 24 hours after each immunization. Rectal temperature was measured with a mouse rectal temperature probe connected to PhysioSuite (Kent Scientific). As shown in FIGS. 13E-13G, FljB significantly increased murine rectal temperature by 0.275° C. at 24 hours after the 1st and 2nd immunizations and increased rectal temperature by 0.625° C. after the 3rd immunization. In contrast, FH VLPs failed to significantly increase rectal temperature after each immunization (FIGS. 13E-13G). These data indicate that the ability to cause elevated rectal temperature in mice is much more reduced in FH VLPs than in FljB. It was further found that FH VLPs and FljB, even at the increased doses, failed to induce microscopic tissue damage or body weight change as compared to PBS-injected mice (FIGS. 14A and 14B), indicating the lack of systemic toxicity in either agent.

### Example 8

### FH VLPs are more Immunogenic than FljB, HBc VLPs or KLH for Nicotine Vaccine Development

The above examples have indicated that FH VLPs might be a more immunogenic and safer carrier than FljB for vaccine development. Next, nicotine vaccines were used as a model to compare relative immunogenicity and safety of FH VLPs to FljB, HBc VLPs, or the widely used keyhole limpet hemocyanin (KLH) as each immunogen was conjugated to nicotine hapten and the resultant complex noted with “-nic”.

Specifically, N-(3-dimethylaminopropyl)-N′-ethylcarbodiimide hydrochloride (EDC) was used to activate the carboxylic acid group of nicotine hapten 6-(carboxymethylureido)-(+/−)-nicotine (CMUNic) for conjugation to primary amine groups of protein immunogens. This procedure is well documented and reported in the art. In more detail, 1 mg CMUNic was activated with 5 mg EDC (E1769, Sigma) in 0.1M 2-(N-morpholino)ethanesulfonic acid (IVIES) buffer (pH4.5) at room temperature for 10 min. FH VLP or other immunogens (1 mg) dissolved 0.1M MES buffer (pH4.5) was added to the above mixture and kept at room temperature for 3 hours with continuous stirring. Nicotine conjugates were then dialyzed against PBS for 3 times to remove unreacted EDC or CMUNic.

Nicotine vaccines hold a great promise for anti-nicotine immunotherapy (T. Raupach, et al., Drugs 2012, 72 (4): el-16). Despite the unsatisfactory clinical trial outcomes for several nicotine vaccines (NicVAX, NicQb, Niccine), different strategies are under exploration to improve anti-nicotine immunotherapy that include making improvement in vaccine carriers (e.g., J.W. Lockner, et al., J. Med. Chem. 2015, 58 (2): 1005-11).

KLH was found to be a more immunogenic carrier than recombinant Pseudomonas Exoprotein A (rEPA) used in NicVAX for nicotine vaccine development (P.T. Bremer, et al., Pharmacol. Rev. 2017, 69(3): 298-315). Flagellin (FliC) was also explored as both vaccine carriers and adjuvants for nicotine vaccine development (N. T. Jacob, et al., J. Med. Chem. 2016, 59 (6): 2523-9). FliC-based nicotine vaccine was found to induce anti-nicotine antibodies with superior nicotine binding to Tetanus Toxoid (TT)-based nicotine vaccine. Here, relative immunogenicity of KLH was first compared to FljB and HBc VLPs for nicotine vaccine development, and tests showed that KLH-Nic elicited —2 times higher anti-nicotine antibody (NicAb) titer than FljB-Nic (data not shown). In another study, relative immunogenicity of KLH-Nic was compared to HBc-Nic, and it was found that KLH-Nic elicited —5 times higher NicAb titer than HBc-Nic (data not shown). These studies indicate that KLH was more immunogenic than FljB and HBc VLPs for nicotine vaccine development.

Next, the relative immunogenicity of FH-Nic was compared to KLH-Nic. Due to the reported synergy between two of the three adjuvants (FljB, CpG, and Alum) (see, e.g., J. W. Lockner, et al., Mol. Pharm. 2015, 12 (2): 653-62), the inventors also evaluated whether incorporation of CpG, Alum, or combinatorial CpG/Alum adjuvant could increase the immunogenicity of FH-Nic. As shown in FIG. 15A, FH-Nic elicited more than 4 times higher NicAb titer than KLH-Nic. Incorporation of CpG or Alum adjuvant further increased NicAb titer by 2.3 and 3.8 times, respectively, while incorporation of CpG/Alum adjuvant failed to further increase NicAb titer as compared to incorporation of either adjuvant alone (FIG. 15A).

Antibody isotype analysis found FH-Nic mainly increased anti-nicotine IgG2a but not IgG1 antibody titer as compared to KLH-Nic (FIGS. 15B and 15C). Incorporation of Thl adjuvant CpG and Th2 adjuvant Alum increased anti-nicotine IgG2a and IgG1 titer, respectively. In line with these data, anti-nicotine IgG2a/IgG1 ratio was increased after incorporation of CpG adjuvant and reduced after incorporation of Alum adjuvant (FIG. 15D). Besides antibody titer, the inventors also evaluated antibody avidity index due to its importance in sequestration of nicotine in the peripheral tissue. Results show that similar antibody avidity index was observed between KLH-Nic and FH-Nic groups. Incorporation of Alum or CpG adjuvant into the FH-Nic vaccination increased antibody avidity to a statistically non-significant level. Interestingly, incorporation of combinatorial CpG/Alum adjuvant failed to increase antibody avidity as compared to FH-Nic vaccination alone (data not shown).

Mice were further challenged with intravenous nicotine and brain and blood were collected 5 minutes later to quantify tissue nicotine levels. As compared to brain nicotine levels of non-immunized mice, KLH-Nic immunization reduced brain nicotine levels by 27% whereas FH-Nic immunization reduced brain nicotine levels by an impressive 63% (FIG. 15E). Incorporation of Alum or CpG adjuvant into FH-Nic immunization reduced brain nicotine levels by 74% and 69%, respectively (FIG. 15E). Incorporation of CpG/Alum combinatorial adjuvant into FH-immunization reduced brain nicotine levels by 70%, similar to incorporation of CpG adjuvant alone (FIG. 15E). Serum nicotine levels showed an opposite trend to brain nicotine levels with relatively big variations within groups (FIG. 15F), in line with other report (V. Arutla, et al., ACS Comb Sci.2017 , 19 (5): 286-298).

Data of all groups were then pooled for linear correlation analysis. A negative correlation was identified between serum NicAb titer and brain nicotine levels (FIG. 15G) and between brain and serum nicotine levels (FIG. 15H), while a positive correlation was found between serum NicAb titer and serum nicotine levels (FIG. 15I). It is believed that the negative correlation between serum NicAb titer and brain nicotine levels supports the generation of high NicAb titer to block nicotine entry into the brain.

Lastly, the inventors explored local and systemic safety of intradermal (ID) FH-Nic immunization and found that ID FH-Nic induced minimal local reactions as supported by the lack of visible changes of FH-Nic-injected skin. ID FH-Nic also failed to induce significant systemic reactions as supported by the baseline levels of serum IL-6 at 6 and 24 hours after immunization (data not shown).

The disclosure of information, methods and data contained in the inventors' publication Y. Zhao et al., Biomaterials, 2020, 249: 120030, is incorporated by reference in its entirety to the extent allowed by the law.

### Example 9

### Construction and Expression of a Recombinant FH-M2e Fusion Protein as a Vaccine Model against Infectious Disease

M2e (influenza matrix protein 2 ectodomain) is highly conserved region among influenza A viruses and has been a highly attractive target for universal influenza vaccine development. Yet, M2e has low immunogenicity and cannot mount potent immune responses. FljB as a potential vaccine carrier has been explored for M2e-based universal influenza vaccine development (FljB-M2e). Yet, FljB-M2e was found to induce systemic adverse reactions in humans due to its overt activation of TLR5 .

The VLPs of the invention (exemplied by FH VLPs) have shown significantly improved immunogenicity and safety as compared to FljB for nicotine vaccine development (Example 8 above). In this example, inventors set up to test the relative immunogenicity and safety of FH VLPs in comparison to FljB and HBc for M2e-based universal influenza vaccine development.

Tandem copies of human Hl/H3, swine H1, and avian H5/H7′s M2e sequences (SEQ ID NOS:19-22), as shown in FIGS. 16B and 16C, were used as vaccine's immunogenic sequence (with optional linkers, e.g., (G4S)×2 linkers, in between segments and at optionally at both ends of the insert to increase protein chain flexibility) to replace the D3 domain of FljB for display on the FH VLP surface (“FH-M2e,” SEQ ID NO:23). Without linkers, the M2e sequence from four influenza variants are 96 amino acid residues long. In one embodiment with linkers, the M2e antigenic sequence inserted into the FH fusion protein is about 146 amino acid residues long.

For comparison purposes, the same sequence of the four tandem copies of M2e variants (with linkers) was used to replace the D3 domain of FljB to generate a recombinant “FljB-M2e” vaccine, and also inserted into the c/el loop of HBc to see whether the resultant “HBc-M2e” fusion protein could self-assemble into VLPs. After recombinant plasmids were constructed, the different immunogens each with a foreign antigen inserted in its sequence (FH-M2e, FljB-M2e, HBc-M2e) were expressed in E. Coli, purified under denaturing conditions, and allowed to refold.

Protein expression was examined in SDS-PAGE (FIG. 16D) and compared with theoretical molecular weight (FIG. 16H), confirming successful expressions. The presence of M2e sequences in purified proteins was then confirmed by blotting the PVDF membrane with immune sera from KLH-M2e immunized mice (FIG. 16E), in which M2e was synthesized by Thermo Fisher Scientific and contained a mixture of 4 different sequences used in this example.

Example 10: FH-M2e but not HBc-M2e self-assembles into VLPs

Transmission electron microscopy (TEM) was used to explore whether FH-M2e and HBc-M2e formed VLPs after refolding. As shown in FIG. 17A, spherical VLPs could be readily identified in FH-M2e but not in HBc-M2e samples. The inability of HBc to form VLPs is in line with the literature that HBc only allows insertion of short antigenic epitopes in order not to jeopardize VLP assembly. This result indicates that FH VLPs constitute a more versatile delivery platform than HBc VLPs for purpose of vaccine development and more.

Dynamic light scattering (DLS) was then used to measure FH-M2e VLP size. As shown in FIG. 17B, FH-M2e has an average particle size of ˜44 nm with a good uniformity as evidenced by the relatively low PDI.

### Example 11

### Efficient Uptake and Stimulation of DC Maturation by FH-M2e VLPs

The uptake ability of the different M2e immunogens by bone marrow derived DCs (BMDCs) were compared. FH-M2e VLPs and HBc-M2e showed a strong uptake, while FljB-M2e and M2e peptide showed a weak uptake by BMDCs (data not shown). Furthermore, significant overlapping of antigen and lysosomal signals was found in FH-M2e VLP and HBc-M2e groups (data not shown), hinting efficient uptake of FH-M2e VLPs and HBc-M2e via endolysosomal pathways.

The ability of the different immunogens to stimulate BMDC maturation was also tested. As shown in FIG. 17C, FH-M2e VLPs and FljB-M2e increased CD40hi, CD80hi, and CD86^{h1} cells to a level similar to that induced by a clinical CpG 1018 adjuvant (simplified as “CpG” sometimes in this disclosure), while HBc-M2e failed to increase CD40hi, CD80hi, and CD86^{h1} cell levels. Statistical analysis found FH-M2e VLPs and FljB-M2e significantly increased MFI of CD40, CD80, and CD86, similarly or slightly exceeding that stimulated by CpG, while HBc-M2e failed to significantly increase MFI of CD40, CD80, or CD86 (upper, FIG. 17D). Similar trends were found about percentage of CD40¹n, CD80¹¹¹, and CD86′' cells (lower, FIG. 17D). These data indicate FH-M2e VLPs can be efficiently taken up by BMDCs and also stimulate potent BMDC maturation (FIG. 17E). In contrast, HBc-M2e can be efficiently taken up but lack the ability to stimulate BMDC maturation, while FljB-M2e can stimulate BMDC maturation but lack the ability of efficient uptake (FIG. 17E). The clear winner here is the VLP of the invention: FH-M2e VLPs.

### Example 12

### Better Immunogenicity and Safety Profile from FH-M2e VLPs

First, the inventors compared relative immunogenicity of FljB-M2e with HBc-M2e, and M2e peptide in the presence of Alum adjuvant (M2e/Alum). As shown in FIG. 18A, FljB-M2e induced the highest anti-M2e antibody titer as compared to HBc-M2e and M2e/Alum. After PR8 viral challenge (4×LD50), mice of all groups lost body weight in the first week due to the lack of neutralizing antibodies (FIG. 18B). Mice in FljB-M2e group started to recover on day 9 and completely recovered on day 14. No mice survived in PBS group and only one out of 4 or 5 mice survived in M2e/Alum and HBc-M2e groups (FIG. 18C). In comparison, 4 out of 5 mice survived in FljB-M2e group.

Serum IL-6 and TNFα levels were also measured at before immunization, 3 hours after, and 18 hours after immunization since both are downstream of TLR5 and linked to systemic adverse reactions of FljB-M2e vaccine in humans. As shown in FIGS. 19A and 19B, FljB-M2e significantly increased serum IL-6 levels at 3 but not 18 hours after prime and boost. HBc-M2e and M2e/Alum immunization failed to significantly increase serum IL-6 levels. FljB-M2e immunization also significantly increased serum TNFα levels at 3 but not 18 hours after prime and boost, while HBc-M2e and M2e/Alum immunizations failed to significantly increase serum TNFα levels (FIGS. 19C and 19D). The above studies indicate that FljB was highly immunogenic for M2e-based universal influenza vaccine development. However, FljB-M2e also induced significant systemic cytokine release, reminiscent of systemic adverse reactions observed in previous clinical studies.

Second, the inventors compared relative immunogenicity of FH-M2e VLPs and FljB-M2e. Furthermore, they also explored whether incorporation of CpG 1018 adjuvant could enhance immunogenicity of FH-M2e VLPs. CpG 1018 is a toll-like receptor (TLR) 9 agonist and has been a clinical adjuvant to boost hepatitis b vaccine efficacy. CpG 1018 is broadly effective in mice, non-human primates, and humans. As shown in FIG. 20A, FH-M2e VLPs induced 2.4-fold higher anti-M2e antibody titer than FljB-M2e. Incorporation of CpG 1018 adjuvant into FH-M2e VLP immunization increased anti-M2e antibody titer by 6.5 folds, while incorporation of CpG 1018 adjuvant into FljB-M2e immunization only increased anti-M2e antibody titer by 1.8 folds.

After PR8 viral challenges (8×LD50), mice in FH-M2e VLP groups showed significantly less body weight loss than that in FljB-M2e group (FIG. 20B). Mice in FH-M2e VLP/CpG and FljB-M2e/CpG groups also showed less body weight loss than that in FH-M2e VLP and FljB-M2e groups, respectively. Overall, mice in FH-M2e VLP/CpG group showed the least body weight loss than other groups. Body weight of mice in FH-M2e VLP/CpG group completely recovered on day 14, while that in FH-M2e VLP and FljB-M2e/CpG groups only partially recovered (-97%). All mice survived in FH-M2e VLP/CpG group and 60% mice survived in FH-M2e VLP group, while no mice survived in FljB-M2e group (FIG. 20C). All mice died in PBS group and 40% mice survived in FljB-M2e VLP/CpG group.

These data strongly indicate the superiority of FH-M2e VLPs (a recombinant embodiment of VLPs of the invention) compared to FljB-M2e in eliciting immune protection against lethal PR8 viral challenges and that the incorporation of CpG could further enhance protective efficacy of FH-M2e VLPs.

The inventors further compared systemic cytokine release among immunization of the different vaccine candidates as a way to further study systemic safety issues. As shown in FIGS. 21A and 21B, FljB-M2e immunization significantly increased serum IL-6 levels at 3 but not 18 hours in both prime and boost, while FH-M2e VLPs failed to significantly increase serum IL-6 levels. Incorporation of CpG didn't further increase serum IL-6 cytokine levels. Similarly, FljB-M2e immunization significantly increased serum TNFα levels at 3 but not 18 hours in both primer and boost, while FH-M2e VLPs failed to increase serum TNFα levels in prime but slightly enhanced serum TNFα levels at 3 hours after boost (FIGS. 21C and 21D). Incorporation of CpG failed to further increase serum TNFα levels. These studies have shown that VLPs of the invention, e.g., FH-M2e, exhibit marked improvement in both immunogenicity and safety as compared to prior art flagellin-based vaccine FljB-M2e, and immunogenicity of VLPs of the invention can be further increased by incorporation of an adjuvant such as CpG.

Body temperature increase was not detected following FljB-M2e immunizations in the above studies. Based on the literature, intradermal immunization is likely to elicit more adverse reactions. To test this, mice were intradermally immunized with FljB-M2e and FH-M2e VLPs. Rectal temperatures of mice were measured before and 24 hours after intradermal immunization. As shown in FIG. 22, intradermal FljB-M2e administration significantly increased rectal temperature of mice, while intradermal administration of FH-M2e VLPs, a vaccine embodiment of the invention, did not significantly increase rectal temperature of mice, indicating the absence of fever.

Example 13

### FH-M2e VLPs Induce Cross Protective Immunity against Various Viral Strains

In this example, any broad cross-protective abilities from FH-M2e VLP or FljB-M2e immunization were evaluated. Mice were prime/boost immunized and then challenged with three different influenza viruses. As shown in FIG. 23A, FH-M2e VLP immunization significantly reduced body weight loss after PR8 virus challenge as compared to PBS group, while FljB-M2e immunization showed a similar trend of body weight loss to PBS group. Surprisingly, FH-M2e VLP conferred 60% protection against lethality, while FljB-M2e failed to confer any protection (FIG. 23A). To test whether FljB-M2e VLPs could elicit superior protection against other viral strains, another immunization study was conducted. Mice were then randomly divided into two groups and challenged with a lethal dose of pdm09 or A/Philippines/2/82 (H3N2) viruses. As shown in FIG. 23B, FH-M2e VLPs (green line) significantly alleviated any body weight loss after pdm09 viral challenges, while FljB-M2e (red line) showed a similar rate of body weight loss to the negative control, the PBS group (blue line). FH-M2e VLPs conferred 75% protection against lethality after pdm09 viral challenges, while FljB-M2e failed to confer any protection (FIG. 23B). After A/Philippines/2/82 (H3N2) viral challenges, FH-M2e VLPs showed a slower rate of body weight loss as compared to FljB-M2e and FH-M2e VLPs conferred 100% protection, while FljB-M2e only conferred 75% protection (FIG. 23C). These data indicate that FH-M2e VLPs could elicit cross-protective immunity against various influenza A viral strains.

In sum, our data support VLPs of the invention to be a more immunogenic, more versatile, and safer carrier for universal influenza vaccine development, at least by incorporating the M2e-immunogenic sequence into the platform, e.g., recombinantly as part of the fusion protein of the invention.

### Example 14

### Construction and Expression of a Recombinant FH-OVA fusion Protein as a Vaccine Model Against Cancer

In a model for cancer vaccine development, the cytotoxic T-lymphocyte (CTL) epitope of ovalbumin (OVA) was recombinantly engineered to replace the highly variable D3 domain of flagellin for display on the FH VLP surface (referred to as “FH-OVA”, SEQ ID NO:24), as schematically shown in FIGS. 16A and 16C. In this and following examples, the same epitope also replaced the D3 domain of flagellin to prepare for a flagellin-based vaccines (referred to as “FljB-OVA”) to compare its immunogenicity and safety profile with the inventive FH VLP-based vaccines. Moreover, the epitope was also inserted into the c/el loop of HBc to explore its impact on HBc VLP assembly and prepare HBc or HBc VLP-based vaccines (referred to as “HBc-OVA”) to compare its immunogenicity with the inventive FH VLP-based vaccines.

For example, OVA₂₄₇-274 (“DEVSGLEQLESIINFEKLTEWTSSNVME”) (SEQ ID NO: 25) encompassing the underlined CTL epitope of OVA₂₅₇-264 (“SIINFEKL”) (SEQ ID NO: 26) in the middle region was used to elicit OVA-specific CTL responses against OVA-expressing E.G7 lymphoma or Bl6F10 melanoma (schematically illustrated in FIG. 16C). To increase protein chain flexibility, (G₄S)x2 linker was added before and after the epitope. Other reference plasmids were constructed using the same CTL epitope.

After all the recombinant plasmids were constructed and OVA-epitope-inserted FljB-HBc, FljB and HBc polypeptides were expressed in E. Coli, purified, and refolded, protein expression was examined in SDS-PAGE and compared with theoretical molecular weight (FIGS. 16F-16H). The gel migration of HBc-OVA, FljB-OVA, and FH-OVA matched very well with the theoretical molecular weight.

FH and HBc samples were further analyzed by TEM and DLS. Successful formation of FH VLP was found after replacement of the D3 domain of FljB with OVA₂₄₇₋₂₇₄ (data not shown). Successful formation of HBc VLP was also found after insertion of OVA₂₄₇₋₂₇₄ into its c/el loop (data not shown). DLS analysis found the OVA-displaying FH VLP to be similar in size as the M2e-displaying FH VLP reported in Example 10, which was bigger than OVA-displaying HBc VLP (data not shown).

### Example 15

### Improved CTL Production and Anti-Tumor Immunity of FH-OVA VLPs as Compared to HBc-OVA VLPs and FljB-OVA

Next, the relative potency of FH-OVA VLPs to induce OVA-specific CTL production was compared with HBc-OVA VLPs and FljB-OVA and also OVA₂₄₇-274 peptide (shorthanded as “OVA”). Due to the low abundance of OVA-specific CD8+ T cells, the inventors adoptively transferred OVA-specific CD8+ T cells isolated from OT-I mice to native C57BL/6 mice followed by intradermal immunization with the different immunogens containing the same amount of OVA₂₄₇-274 peptide. Draining lymph nodes were harvested 4 days later and CF SE+cells in CD8+ T cells were analyzed.

As shown in FIGS. 24A and 24B, FH-OVA VLP immunization induced the most significant expansion of adoptively transferred OT-I cells, followed by HBc VLP immunization, and then FljB-OVA and OVA peptide immunization. Statistical analysis indicated FH-OVA VLPs were more potent than HBc-OVA VLPs and FljB-OVA to stimulate adoptively transferred OT-I cell expansion (FIG. 24B).

Anti-tumor immunity was then compared following three repeated immunization with the different immunogens followed by challenging mice with OVA-expressing E.G7 lymphoma or Bl6F10 melanoma. Tumor growth and survival of tumor-bearing mice were monitored and compared among groups. FH-OVA VLP immunization most significantly retarded E.G7-OVA lymphoma growth with tumor volume significantly smaller than that in non-immunized group on day 13 and 16. In contrast, HBc-OVA VLP, FljB-OVA, and OVA peptide immunization failed to significantly reduce E.G7-OVA lymphoma growth with tumor volume at each time point showed no significant difference from that in non-immunized group (FIG. 24C). FH-OVA immunization also significantly extended survival of E.G7-OVA-bearing mice, while the other immunizations failed to do so (FIG. 24D).

Besides OVA-expression E.G7 lymphoma model, immunized mice were also challenged with OVA-expressing Bl6F10 melanoma in a different study. As shown in FIG. 24E, FH-OVA VLP immunization most significantly reduced Bl6F10 melanoma growth, followed by HBc-OVA VLP and FljB-OVA immunization. Tumor volume was significantly smaller on days 13 and 16 in FH-OVA VLP group as compared to that in non-immunized group. Tumor volume was also significantly smaller on day 16 in FH-OVA VLP group than that in FljB-OVA group (FIG. 24E). Tumor volume in HBc-OVA VLP group was also significantly smaller on day 13 and 16 than that in non-immunized group (FIG. 24E). Tumor volume in FljB-OVA group was significantly smaller on day 16 than that in non-immunized group (FIG. 24E).

FH-OVA VLP immunization also significantly extended survival of Bl6F10-OVA-bearing mice, while HBc-OVA VLP immunization failed to significantly extend survival of N₁₆F10-OVA-bearing mice (FIG. 24F). Interestingly, FljB-OVA also significantly extended survival of Bl6F10-OVA-bearing mice (FIG. 24F), while such an effect was not as significant as with FH-OVA VLP immunization.

The above results indicate that FH VLPs provided the most immunogenic carrier to develop cancer vaccines to elicit the most potent CTL production and anti-tumor immunity, as compared to HBc VLPs and FljB. Although only a short CTL epitope was used as a model in the current example, longer antigenic epitopes and full-length neoantigens can be readily inserted for display on FH VLPs to elicit potent anti-tumor immunity as D3 domain of FljB allows insertion of diverse vaccine antigens with little restriction on length or 3D structure.

Although inventors mainly focused on exploring CTL and anti-tumor responses, anti-OVA₂₄₇-274 antibody responses were also measured after the completion of the last immunization. In those studies, FljB-OVA and FH-OVA VLP but not HBc-OVA VLP induced significantly higher anti-OVA IgG titer as compared to OVA immunization alone (data not shown). Further, it was found that FljB-OVA induced significantly higher IgG1 titer, while FH-OVA VLP induced significantly higher IgG2c titer, as compared to OVA immunization alone (data not shown). This indicates that FH-OVA VLP induced mainly Thl-biased antibody responses, while FljB-OVA mainly induced Th2-biased antibody responses.

Furthermore, PBMCs were prepared 7 days after the last immunization and stimulated with synthetized OVA₂₄₇-274 peptide followed by intracellular cytokine staining and flow cytometry analysis. HBc-OVA and FH-OVA VLP immunization induced the most significant increase of IFNy-secreting CD8+ T cells, while FH-OVA VLP but not HBc-OVA VLP immunization significantly increased IL4-secreting CD8+ T cells. These data support the high potency of FH-OVA VLPs to induce IFNy and IL4-secreting CTLs. Furthermore, FH-OVA VLP but not HBc-OVA VLP or other immunizations significantly increased IFNy and IL4-secreting CD4+ T cells (data not shown). Significant increase of CD4+ helper T cells was in line with the induction of highest anti-OVA antibody titer in FH-OVA VLP group.

Example 16: CpG 1018 boosts FH-OVA VLP immunization

In this example, inventors explored whether incorporation of CpG 1018, a Th1 adjuvant, could further enhance CTL production and anti-tumor immunity of FH-OVA VLPs.

Adoptive transfer and immunization studies were first conducted to evaluate OT-I cell expansion as well as Granzyme B, TNFa, and IFNy expression due to crucial roles of these cytokines in anti-tumor immunity. As shown in FIG. 25A, incorporation of CpG didn't further enhance OT-I cell expansion. However, incorporation of CpG significantly increased single, dual, and triple cytokine-secreting OT-I cells (FIG. 25A).

Mice were then immunized with FH-OVA VLPs in the presence or absence of CpG 1018 or PBS to serve as control. Immunization was repeated three times and one week after the last immunization, PBMCs were collected, stimulated with CTL epitope of OVA, and cytokine-secreting OVA-specific CD8+ T cells were then analyzed. Incorporation of CpG 1018 into FH-OVA VLP immunization could significantly enhance OVA-specific single, dual, and triple cytokine-secreting CD8+ T cells, in line with the above adoptive transfer study (data not shown). Although inventors mainly focused on exploring CTL responses in this study, anti-OVA₂₄₇-274 IgG, subtype IgG1 and IgG2c antibody responses were also measured. Results showed that CpG 1018 adjuvant significantly increased IgG2c but not IgG1 antibody production. The ratio of IgG2c to IgG1 was significantly increased by incorporation of CpG 1018 adjuvant (data not shown), hinting the induction of a Thl-biased antibody responses. CpG 1018 also increased total IgG level to a non-statistically significant level due to the large variations of IgG titer in both groups (data not shown).

Mice were then challenged with Bl6F10-OVA melanoma. As shown in FIG. 25B, FH-OVA VLP immunization alone significantly retarded tumor growth and incorporation of CpG 1018 further amplified the anti-tumor effects. Specifically, FH-OVA VLP immunization in the presence of CpG prevented tumor growth in 20% of mice (FIG. 25C). FH-OVA VLP immunization significantly extended survival of tumor-bearing mice, while incorporation of CpG 1018 more significantly extended survival of tumor-bearing mice (FIG. 25D). These results indicate that CpG 1018 adjuvant was highly potent to further enhance anti-tumor efficacy of the VLPs of the invention, e.g., the FH-OVA VLPs.

Example 17: Better systemic safety of FH VLP-based vaccines as compared to flagellin-based vaccines

Besides evaluation of the immunogenicity and protective efficacy, systemic safety of FH VLP-based vaccines was also tested and compared against flagellin-based vaccines. Since over-activation of TLR5 was likely contributed to the observed systemic safety of flagellin-based vaccines, inventors mainly measured serum IL-6 and TNFα levels as these two cytokines are downstream of TLR5 signaling pathways and linked to systemic adverse reactions of flagellin-based vaccines in humans.

In Example 12 above, inventors found FH-M2e VLPs did not significantly increase serum IL-6 levels at 3 hours in either prime or boost immunization and only slightly increased serum TNFα levels at 3 hours in boost but not prime immunization (FIGS. 21A-D). Further, incorporation of CpG 1018 in FljB-M2e or FH-M2e VLP immunization didn't further enhance serum IL-6 or TNFα levels (FIGS. 21A-D), meaning that the safety profile remained sound with co-administration of the adjuvant. Finally, in tests of rectal temperature of immunized mice, FH-M2e VLP (33.2 pg) immunization, unlike flagellin-based counterpart (FljB-M2e), did not significantly increase rectal temperature of tested mice (FIGS. 21E and 21F). The above results indicated FH-M2e VLPs had a good systemic safety, while FljB-M2e had a high risk to induce systemic adverse reactions.

Besides M2e-based universal influenza vaccines, inventors also compared systemic cytokine release following three immunizations of OVA-based cancer vaccines in this example. FljB-OVA induced the most significant increase of serum IL-6 and TNFα levels at 3 hours following each immunization, while FH-OVA VLP immunization did not significantly increase serum IL-6 and TNFα levels (FIGS. 26A and 26B). Due to the crucial roles of TLR5 in systemic adverse reactions of flagellin-based vaccines, we also analyzed TLR5 activation ability of FljB-OVA and FH-OVA VLPs as well as FljB D3 domain-deleted FH VLPs. The data show that FljB-OVA similarly activated TLR5 as compared to native FljB, indicating that replacement of the D3 domain with OVA peptide did not significantly impact TLR5 activation ability. FH-OVA VLPs showed much weakened TLR5 activation ability as compared to FljB-OVA and similar TLR5 activation ability as compared to FljB D3 domain-depleted FH VLPs (data not shown). Furthermore, we found incorporation of 40₁.tg CpG 1018 into FH-OVA VLP immunization significantly increased serum IL-6 and TNFα levels at 3 hours (data not shown). However, such an increase was much less than that induced by FljB-OVA (FIGS. 26A and 26B). For example, serum IL-6 level following the 1st immunization by FH-OVA VLPs, which induced the most significant release of systemic cytokines, was only 18% of that induced by the 1st immunization by FljB-OVA (FIG. 26A). These results indicated although incorporation of 40₁.tg of CpG 1018 into FH-OVA VLP immunization increased systemic cytokine release, the risk to induce systemic adverse reactions was much less in comparison to flagellin-based vaccines alone.

The generation of FH VLP-bases vaccines involves construction of recombinant plasmid to express FljB-HBc fusion proteins, in which FljB is inserted into c/el loop of HBc and D3 domain of FljB is replaced with vaccine antigens, followed by E. Coli expression, purification, and self-assembly into VLPs (FIG. 16A). FH VLP-based vaccines are proven to have a good immunogenicity, capable of eliciting both humoral and cellular immune responses, due to the ability of VLP platform to target vaccine antigens to major histocompatibility complex (MEW) class I presentation pathway and the stimulation of DC maturation by FljB adjuvant. FH VLP-based vaccines also have a good safety due to the embedding of TLR5 activation site in the interior of VLPs and over-activation of TLR5 has contributed to systemic adverse reactions of Flagellin-based vaccines. D3 domain can be replaced with diverse vaccine antigens without affecting VLP assembly and thus FH VLPs might provide a versatile platform for vaccine development. Further, incorporation of an adjuvant, e.g., a clinical CpG 1018 adjuvant, can further enhance FH VLP-based vaccine efficacy.

While the present invention has been particularly shown and described with reference to the structure and methods disclosed herein and as illustrated in the drawings, it is not confined to the details set forth and this invention is intended to cover any modifications and changes as may come within the scope and spirit of the following claims. All publications and patent literature described herein are incorporated by reference in entirety to the extent permitted by applicable laws and regulations.

1. A fusion protein comprising (a) an amino acid sequence for at least both the D0 and D1 domains of a flagellin protein or substantial portions thereof, and (b) an amino acid sequence for a polypeptide that would, in aggregate, form a virus-like particle (VLP) (a “VLP-forming polypeptide”), wherein (a) and (b) are recombinantly linked to each other.

2. The fusion protein of claim 1, wherein the fusion protein, together with other such fusion proteins, self-assemble to form a VLP in an aqueous environment.

3. The fusion protein of claim 1 wherein the VLP-forming polypeptide comprises a Hepatitis B core (HBc) protein or a substantial portion thereof.

4. The fusion protein of claim 3 wherein (a) is recombinantly inserted into the amino acid sequence of the c/el loop (N75-L84) of the HBc protein or the substantial portion thereof.

5. The fusion protein of claim 3, wherein (a) is recombinantly inserted to replace part or all of the c/el loop (N75-L84) of the HBc protein or the substantial portion thereof.

6. The fusion protein of claim 1 wherein the flagellin protein is selected from the group consisting of FljB, FliC, FlpA, FlaA, and FlaB variants.

7. The fusion protein of claim 1 wherein the flagellin protein is native to the group of flagellated bacteria consisting of Salmonella, Escherichia coli, Vibrio vulnificus, Campylobacter coli, Bacillus subtilis, Burkholderia pseudomallei and Pseudomonas aeruginosa.

8. The fusion protein of claim 1 comprising the amino acid sequence of at least highly conserved regions of N and C termini of flagellin variants (SEQ ID NO: 3) or substantial portions thereof.

9. The fusion protein of claim 1 further comprising an amino acid sequence for the D2 domain of the flagellin or a substantial portion thereof.

10. The fusion protein of claim 1 further comprising an amino acid sequence for the D3 domain of the flagellin or a substantial portion thereof.

11. The fusion protein of claim 1 further comprising (c) an immunogenic sequence.

12. The fusion protein of claim 11 wherein the immunogenic sequence comprises an epitope or a full-length antigen.

13. The fusion protein of claim 12 wherein the epitope or full-length antigen comprises a portion of an amino acid sequence of an influenza protein.

14. The fusion protein of claim 13, wherein the portion of the influenza protein comprises the ectodomain of influenza matrix protein 2 (M2e).

15. The fusion protein of claim 12 wherein the epitope or full-length antigen comprises multiple copies of the ectodomain of influenza matrix protein 2 (M2e).

16. The fusion protein of claim 12 wherein the epitope or full-length antigen comprises a portion of the ovalbumin (OVA) protein.

17. The fusion protein of claim 12 portion of the OVA protein comprises a tumor-associated antigen (TAA) or a neoantigen.

18. The fusion protein of claim 11 wherein the immunogenic sequence is recombinantly inserted into the amino acid sequence for the D2 or D3 domain of flagellin.

19. The fusion protein of claim 11 wherein the immunogenic sequence is recombinantly inserted into the fusion protein by replacing part or all of the D2 domain, D3 domain or both D2 and D3 domains of flagellin.

20. The fusion protein of claim 1, further comprising a linker sequence between (a) and (b).

21. The fusion protein of claim 1, used as a vaccine, a vaccine carrier or a vaccine adjuvant.

22-26. (canceled)

27. A fusion protein comprising amino acid sequences for (a) an antigen, (b) a flagellin protein or a substantial portion thereof, (c) a virus-like particle (VLP)-forming polypeptide, wherein (a) (b) and (c) are recombinantly linked.

28-46. (canceled)


SEQUENCE LISTING (29 sequences)

SEQ ID NO: 1 - protein, 506 aa, S. Typhimurium
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVQKAYDVKDTAVTTKAYANNGTTLDVSGLDDAAIKAATGGTNGTASVTGGAVKFDADNNKYFVTIGGFTGADAAKNGDYEVNVATDGTVTLAAGATKTTMPAGATTKTEVQELKDTPAVVSADAKNALIAGGVDATDANGAELVKMSYTDKNGKTIEGGYALKAGDKYYAADYDEATGAIKAKTTSYTAADGTTKTAANQLGGVDGKTEVVTIDGKTYNASKAAGHDFKAQPELAEAAAKTTENPLQKIDAALAQVDALRSDLGAVQNRFNSAITNLGNTVNNLSEARSRIEDSDYATEVSNMSRAQILQQAGTSVLAQANQVPQNVLSLLR

SEQ ID NO: 2 - protein, 495 aa, S. Typhimurium [sequence omitted]

SEQ ID NO: 3 - protein, 391 aa, artificial
  Consensus sequence
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDLNVQYVDTATYATTLDKAGGTGKFDKYVTGTGKGYEVVTGVTLAGATPAATKVADAKALAGVTAVKMSYTDNGKTIGGAKGDYYAGITTYTADGTKTANLGGDGKTEVVIGKTYASKAGHFKAQPLAEAAATTENPLQKIDAALAQVDLRSDLGAVQNRFNSAITNLGNTVNNLARSRIEDSDYATEVSNMSRAQILQQAGTSVLAQANQVPQNVLSLLR

SEQ ID NO: 4 - protein, 42 aa, S. Typhimurium
MAQVINTNSLSLLTQNNLNKSQSALGTAIERLSSGLRINSAK

SEQ ID NO: 5 - protein, 51 aa, S. Typhimurium
  misc_feature (4)..(5): Xaa can be any naturally occurring amino acid
NNLXXARSRIEDSDYATEVSNMSRAQILQQAGTSVLAQANQVPQNVLSLLR

SEQ ID NO: 6 - protein, 138 aa, S. Typhimurium
  misc_feature (130)..(130): Xaa can be any naturally occurring amino acid
  misc_feature (135)..(136): Xaa can be any naturally occurring amino acid
  misc_feature (138)..(138): Xaa can be any naturally occurring amino acid
DDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDXLNVQXXYX

SEQ ID NO: 7 - protein, 58 aa, S. Typhimurium
  misc_feature (1)..(1): Xaa can be any naturally occurring amino acid
  misc_feature (4)..(4): Xaa can be any naturally occurring amino acid
  misc_feature (10)..(10): Xaa can be any naturally occurring amino acid
  misc_feature (17)..(17): Xaa can be any naturally occurring amino acid
  misc_feature (35)..(35): Xaa can be any naturally occurring amino acid
XGHXFKAQPXLAEAAAXTTENPLQKIDAALAQVDXLRSDLGAVQNRFNSAITNLGNTV

SEQ ID NO: 8 - protein, 573 aa, Salmonella typhimurium
MGFRINTNVAALNAKANSDLNSRALDQSLSRLSSGLRINSAADDASGMAIADSLRSQANTLGQAISNGNDALGILQTADKAMDEQLKILDTIKTKATQAAQDGQSLKTRTMLQADINRLMEELDNIANTTSFNGKQLLSGGFTNQEFQIGSSSNQTIKASIGATQSSKIGVTRFETGSQSFSSGTVGLTIKNYNGIEDFKFQNVVISTSVGTGLGALAEEINRNADKTGIRATFDVKSVGAYAIKAGNTSQDFAINGVVIGKVDYSDGDENGSLISAINAVKDTTGVQASKDENGKLVLTSADGRGIKITGSIGVGAGILHTENYGRLSLVKNDGRDINISGTGLSAIGMGATDMISQSSVSLRESKGQISAANADAMGFNAYNGGGAKQIIFASSIAGFMSQAGSGFSAGSGFSVGSGKNYSAILSASIQIVSSARSISSTYVVSTGSGFSAGSGNSQFAALRISTVSAHDETAGVTTLKGAMAVMDIAETAITNLDQIRADIGSVQNQITSTINNITVTQVNVKSAESQIRDVDFASESANYSKANILAQSGSYAMAQANSSQQNVLRLLQ

SEQ ID NO: 9 - protein, 482 aa, Salmonella typhimurium
MAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDTLNVQQKYKVSDTAATVTGYADTTIALDNSTFKASATGLGGTDQKIDGDLKFDDTTGKYYAKVTVTGGTGKDGYYEVSVDKTNGEVTLAGGATSPLTGGLPATATEEAKAALTAAGVTGTASVVKMSYTDNNGKTIDGGLAVKVGDDYYSATQNKDGSISINTTKYTADDGTSKTALNKLGGADGKTEVVSIGGKTYAASKAEGHNFKAQPDLAEAAATTTENPLQKIDAALAQVDTLRSDLGAVQNRFNSAITNLGNTVNNLTSARSRIEDSDYATEVSNMSRAQILQQAGTSVLAQANQVPQNVLSLLR

SEQ ID NO: 10 - protein, 465 aa, Salmonella danysz
AQVINTNSLSLLTQNNLNKSQSSLSSAIERLSSGLRINSAKDDAAGQAIANRFTSNIKGLTQASRNANDGISIAQTTEGALNEINNNLQRVRELSVQATNGTNSDSDLKSIQDEIQQRLEEIDRVSNQTQFNGVKVLSQDNQMKIQVGANDGETITIVLQKIDVKSLGLDGFNVNGPKEATVGDLKSSFKNVTGYDTYAAGADKYRVDINSGAVVTDAAAPDKVYVNAANGQLTTDDAENNTAVDLFKTTKSTAGTAEAKAIAGAIKGGKEGDTFDYKGVTFTIDTKTGNDGNGKVSTTINGEKVTLTVADIATGATDVNAATLQSSKNVYTSVVNGQFTFDDKTKNESAKLSDLEANNAVKGESKITVNGAEYTANPLASIDSALSKVDAVRSSLGAIQNRFDSAITNLGNTVTNLNSARSRIEDADYATEVSNMSKAQILQQAGTSVLAQANQVPQNVLSLLR

SEQ ID NO: 11 - protein, 573 aa, C.coli; identical to SEQ ID NO: 8

SEQ ID NO: 12 - protein, 394 aa, Pseudomonas aeruginosa
MALTVNTNIASLNTQRNLNNSSASLNTSLQRLSTGSRINSAKDDAAGLQIANRLTSQVNGLNVATKNANDGISLAQTAEGALQQSTNILQRMRDLSLQSANGSNSDSERTALNGEAKQLQKELDRISNTTTFGGRKLLDGSFGVASFQVGSAANEIISVGIDEMSAESLNGTYFKADGGGAVTAATASGTVDIAIGITGGSAVNVKVDMKGNETAEQAAAKIAAAVNDANVGIGAFSDGDTISYVSKAGKDGSGAITSAVSGVVIADTGSTGVGTAAGVAPSATAFAKTNDTVAKIDISTAKALSRRAGDRTTAIKQIDASVPTSVAVQNRFDNTINNLKNIGENVSAARGRIEDTDFAAETANLTKNQVLQQAGTAILAQANQLPQSVLSLLR

SEQ ID NO: 13 - protein, 488 aa, Pseudomonas aeruginosa
MALTVNTNIASLNTQRNLNASSNDLNTSLERLTTGYRINSAKDDAAGLQISNRLSNQISGLNVATRNANDGISLAQTAEGALQQSTNILQRIRDLALQSANGSNSDADRAALQKEVAAQQAELTRISDTTTFGGRKLLDGSFGTTSFQVGSNAYETIDISLQNASASAIGSYQVGSNGAGTVASVAGTATASGIASGTVNLVGGGQVKNIAIAAGDSAKAIAEKMDGAIPNLSARARTVFTADVSGVTGGSLNFDVTVGSNTVSLAGVTSTQDLADQLNSNSSKLGITASINDKGVLTITSATGENVKFGAQTGTATAGQVAVKVQGSDGKFEAAAKNVVAAGTAATTTIVTGYVQLNSPTAYSVSGTGTQASQVFANASAAQKSSVASVDISTADGAQNAIAVVDNALAAIDAQRADLGAVQNRFKNTIDNLTNISENATNARSRIKDTDFAAETAALSKNQVLQQAGTAILAQANQLPQAVLSLLR

SEQ ID NO: 14 - protein, 563 aa, Escherichia coli
MAQVINTNSLSLITQNNINKNQSALSSSIERLSSGLRINSAKDDAAGQAIANRFTSNIKGLTQAARNANDGISVAQTTEGALSEINNNLQRVRELTVQATTGTNSQSDLDSIQDEIKSRLDEIDRVSGQTQFNGVNVLAKDGSMKIQVGANDGQTITIDLKKIDSSTLNLTGFNVNGSGSVANTAATKADLTAAQLSAPGAADANGTVTYTVSAGYKESTAADVIASIKDGSAPTSAITATINNGFGDSSALTSNDYTYDPAKGDFTYDVASSANNTAAQVQSFLTPKAGDTANLKVTVGTTSVDVVLASDGKITAKDGSALYIDSTGNLTQNSAGLTSAKLATLTGLQGSGVASTITTEDGTNIDIAANGNIGLTGVRISADSLQSATKSTGFTVGTGATGLTVGTDGKVTIGGTTAQSYTSKDGSLTTDNTTKLYLQKDGSVTNGSGKAVYVEADGDFTTDAATKAATTTDPLKALDEAISQIDKFRSSLGAIQNRLDSAVTNLNNTTTNLSEAQSRIQDADYATEVSNMSKAQIIQQAGNSVLAKANQVPQQVLSLLQGP

SEQ ID NO: 15 - protein, 10 aa, Hepatitis B
NLEDPASREL

SEQ ID NO: 16 - protein, 149 aa, Hepatitis B
MDIDPYKEFGASVELLSFLPSDFFPSIRDLLDTASALYREALESPEHCSPHHTALRQAILCWGELMNLATWVGSNLEDPASRELVVSYVNVNMGLKIRQLLWFHISCLTFGRETVLEYLVSFGVWIRTPPAYRPPNAPILSTLPETTVV

SEQ ID NO: 17 - protein, 183 aa, Hepatitis B
MDIDPYKEFGASVELLSFLPSDFFPSIRDLLDTASALYREALESPEHCSPHHTALRQAILCWGELMNLATWVGSNLEDPASRELVVSYVNVNMGLKIRQLLWFHISCLTFGRETVLEYLVSFGVWIRTPPAYRPPNAPILSTLPETTVVRRRGRSPRRRTPSPRRRRSQSPRRRRSQSRESQC

SEQ ID NO: 18 - protein, 10 aa, Artificial
  linker sequence
GGGGSGGGGS

SEQ ID NO: 19 - protein, 24 aa, Human influenza H1/H3
SLLTEVETPIRNEWGCRCNDSSDP

SEQ ID NO: 20 - protein, 24 aa, Swine influenza H1
SLLTEVETPTRSEWECRCSDSSDP

SEQ ID NO: 21 - protein, 24 aa, Avian influenza H5
SLLTEVETPTRNEWECRCSDSSDP

SEQ ID NO: 22 - protein, 24 aa, Avian influenza H7
SLLTEVETPTRTGWECNCSGSSEP

SEQ ID NO: 23 - protein, 727 aa, Artificial
  fusion protein
MDIDPYKEFGASVELLSFLPSDFFPSIRDLLDTASALYREALESPEHCSPHHTALRQAILCWGELMNLATWVGSNLEDPAGGGGSGGGGSAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVQKAYDVKDTAVTTKAYGGGGSGGGGSSLLTEVETPIRNEWGCRCNDSSDPGGGGSGGGGSSLLTEVETPTRSEWECRCSDSSDPGGGGSGGGGSSLLTEVETPTRNEWECRCSDSSDPGGGGSGGGGSSLLTEVETPTRTGWECNCSGSSEPGGGGSGGGGSVVSADAKNALIAGGVDATDANGAELVKMSYTDKNGKTIEGGYALKAGDKYYAADYDEATGAIKAKTTSYTAADGTTKTAANQLGGVDGKTEVVTIDGKTYNASKAAGHDFKAQPELAEAAAKTTENPLQKIDAALAQVDALRSDLGAVQNRFNSAITNLGNTVNNLSEARSRIEDSDYATEVSNMSRAQILQQAGTSVLAQANQVPQNVLSLLRGGGGSGGGGSSRELVVSYVNVNMGLKIRQLLWFHISCLTFGRETVLEYLVSFGVWIRTPPAYRPPNAPILSTLPETTVVLEHHHHHH

SEQ ID NO: 24 - protein, 629 aa, Artificial
  man-made fusion protein
MDIDPYKEFGASVELLSFLPSDFFPSIRDLLDTASALYREALESPEHCSPHHTALRQAILCWGELMNLATWVGSNLEDPAGGGGSGGGGSAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVQKAYDVKDTAVTTKAYGGGGSGGGGSDEVSGLEQLESIINFEKLTEWTSSNVMEGGGGSGGGGSVVSADAKNALIAGGVDATDANGAELVKMSYTDKNGKTIEGGYALKAGDKYYAADYDEATGAIKAKTTSYTAADGTTKTAANQLGGVDGKTEVVTIDGKTYNASKAAGHDFKAQPELAEAAAKTTENPLQKIDAALAQVDALRSDLGAVQNRFNSAITNLGNTVNNLSEARSRIEDSDYATEVSNMSRAQILQQAGTSVLAQANQVPQNVLSLLRGGGGSGGGGSSRELVVSYVNVNMGLKIRQLLWFHISCLTFGRETVLEYLVSFGVWIRTPPAYRPPNAPILSTLPETTVVLEHHHHHH

SEQ ID NO: 25 - protein, 28 aa, Chicken
DEVSGLEQLESIINFEKLTEWTSSNVME

SEQ ID NO: 26 - protein, 8 aa, Chicken
SIINFEKL

SEQ ID NO: 27 - protein, 682 aa, Artificial
  Man-made fusion protein
MDIDPYKEFGASVELLSFLPSDFFPSIRDLLDTASALYREALESPEHCSPHHTALRQAILCWGELMNLATWVGSNLEDPAGGGGSGGGGSAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVQKAYDVKDTAVTTKAYANNGTTLDVSGLDDAAIKAATGGTNGTASVTGGAVKFDADNNKYFVTIGGFTGADAAKNGDYEVNVATDGTVTLAAGATKTTMPAGATTKTEVQELKDTPAVVSADAKNALIAGGVDATDANGAELVKMSYTDKNGKTIEGGYALKAGDKYYAADYDEATGAIKAKTTSYTAADGTTKTAANQLGGVDGKTEVVTIDGKTYNASKAAGHDFKAQPELAEAAAKTTENPLQKIDAALAQVDALRSDLGAVQNRFNSAITNLGNTVNNLSEARSRIEDSDYATEVSNMSRAQILQQAGTSVLAQANQVPQNVLSLLRGGGGSGGGGSSRELVVSYVNVNMGLKIRQLLWFHISCLTFGRETVLEYLVSFGVWIRTPPAYRPPNAPILSTLPETTVVLEHHHHHH

SEQ ID NO: 28 - protein, 448 aa, Artificial
  Man-made fusion protein
MDIDPYKEFGASVELLSFLPSDFFPSIRDLLDTASALYREALESPEHCSPHHTALRQAILCWGELMNLATWVGSNLEDPAGGGGSGGGGSAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLLAEAAAKTTENPLQKIDAALAQVDALRSDLGAVQNRFNSAITNLGNTVNNLSEARSRIEDSDYATEVSNMSRAQILQQAGTSVLAQANQVPQNVLSLLRGGGGSGGGGSSRELVVSYVNVNMGLKIRQLLWFHISCLTFGRETVLEYLVSFGVWIRTPPAYRPPNAPILSTLPETTVVLEHHHHHH

SEQ ID NO: 29 - protein, 581 aa, Artificial
  Man-made fusion protein
MDIDPYKEFGASVELLSFLPSDFFPSIRDLLDTASALYREALESPEHCSPHHTALRQAILCWGELMNLATWVGSNLEDPAGGGGSGGGGSAQVINTNSLSLLTQNNLNKSQSALGTAIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVQKAYDVKDTAVTTKAYVVSADAKNALIAGGVDATDANGAELVKMSYTDKNGKTIEGGYALKAGDKYYAADYDEATGAIKAKTTSYTAADGTTKTAANQLGGVDGKTEVVTIDGKTYNASKAAGHDFKAQPELAEAAAKTTENPLQKIDAALAQVDALRSDLGAVQNRFNSAITNLGNTVNNLSEARSRIEDSDYATEVSNMSRAQILQQAGTSVLAQANQVPQNVLSLLRGGGGSGGGGSSRELVVSYVNVNMGLKIRQLLWFHISCLTFGRETVLEYLVSFGVWIRTPPAYRPPNAPILSTLPETTVVLEHHHHHH